(19) United States
(12) Patent Application Publication
(10) Pub. No.: US 2018/0031584 A1
(43) Pub. Date: Feb. 1, 2018

(54) METHODS OF DETECTING ANALYTES AND DIAGNOSING TUBERCULOSIS

(71) Applicant: UNIVERSITY OF UTAH RESEARCH FOUNDATION, Salt Lake City, UT (US)

(72) Inventors: Marc D. PORTER, Cottonwood Heights, UT (US); Jennifer H. GRANGER, Salt Lake City, UT (US); Alexis C. CRAWFORD, Murray, UT (US); Lars Bjorn LAURENTIUS, Salt Lake City, UT (US)

(21) Appl. No.: 15/550,183

(22) PCT Filed: Feb. 10, 2016
(86) PCT No.: PCT/US2016/017287

§ 371 Date: Aug. 10, 2017

Related U.S. Application Data
(60) Provisional application No. 62/114,491, filed on Feb. 10, 2015.

(51) Int. Cl. G01N 33/92; G01N 33/569

(52) U.S. Cl. CPC G01N 33/92; G01N 33/5695; G01N 2333/35; G01N 2405/00

(57) ABSTRACT

A method for detecting lipoarabinomannan in a biological sample is described, including the step of contacting the biological sample with at least one acid selected from the group consisting of perchloric acid, trifluoroacetic acid, and sulfosalicylic acid. Methods for diagnosing diseases, including tuberculosis, and kits for the described methods are also presented.

### CROSS REFERENCE TO RELATED APPLICATIONS

This application claims the benefit of U.S. Provisional Patent Application Ser. No. 62/114,491, filed on Feb. 10, 2015, which is hereby incorporated by reference in its entirety for all of its teachings.

### STATEMENT REGARDING FEDERALLY FUNDED RESEARCH

This invention was made with government support under U18-FD004034-01 awarded by the U.S. Food and Drug Administration. The government has certain rights in the invention.

### FIELD OF INVENTION

The disclosure provided herein relates to methods of detecting analytes that include a novel pretreatment step, methods of diagnosing disease including tuberculosis, and kits incorporating the same.

### BACKGROUND

Diagnostic tests for diseases such as tuberculosis (TB) are critical for patient care and global infection control. An antigen useful for TB detection is lipoarabinomannan (LAM), a lipoglycan unique to mycobacteria. This disclosure describes the development and validation of methods useful for patient serum testing which use surface-enhanced Raman scattering (SERS) or an enzyme-linked immunosorbent assay (ELISA) for the detection of the exemplary analyte LAM.

The methods developed and described herein for preparing the serum sample are amenable to other analyte detection technologies as well, encompassing essentially any assay or analytical process which provides a signal or response to a change or the presence (or absence) of an analyte. These platforms include, for example, ELISA, surface-enhanced Raman scattering (SERS)-based immunoassay, any assay using detection via fluorescence, diffuse reflectance, mass spectrometric, liquid or gas chromatographic spectroscopies, any magnetic, colorometric or electrochemical based detection platform, lateral and vertical flow assays, and kinetics-based assays such as surface plasmon resonance.

Advances in TB diagnostics stand as one of the major priorities in global health. TB is the world's second deadliest infectious disease. The challenges associated with combatting this disease are amplified by the emergence of drug-resistant strains of Mycobacterium tuberculosis (Mtb) and by individuals co-infected with human immunodeficiency virus (HIV). The World Health Organization (WHO) estimates that there were 8.6 million active cases of TB in 2012 and 1.4 million associated deaths; the majority (˜80%) of these cases occurred in resource-limited countries.

If detected early, TB can be cured. Early detection is also vital in containing the spread of the disease. However, sputum smear microscopy (SSM), the test most widely available in resource-limited areas of the world, cannot reliably detect early-stage infection. Serological diagnostics have also proven ineffective in cases in which an individual is immunocompromised by HIV co-infection, due, in large part, to the inability of the patient to generate antibodies. Nucleic acid amplification tests (NAATs) can be of value in early diagnosis, but are only now being engineered and tested in formats that may potentially meet the requirements (e.g., low cost, short turn-around-time, and ease-of-use) of TB-endemic settings.

In recognition of these challenges, there has been a refocus in TB diagnostics toward the direct detection of primary antigenic markers of Mtb in serum and other body fluids. This strategy parallels a proven approach for the early diagnosis of malaria and other diseases. The potential merits of this strategy include: (1) high clinical sensitivity and specificity; (2) direct quantifiable evidence of the disease; (3) diagnosis of smear-negative pulmonary infection; and (4) lack of dependence on a functioning immune system. Serum and urine assays may also be useful in diagnosing extrapulmonary TB. This form of TB is a common and difficult-to-detect form of the disease often found in children, who may be unable to produce sputum, and in HIV co-infected adults.

Several mycobacterial antigens have been found in serum and other body fluids (e.g., urine, sputum, and cerebral spinal fluid) of TB-infected patients. The most widely investigated antigen for use in TB diagnostics is lipoarabinomannan (LAM), a 17.5 kDa lipoglycan unique to mycobacteria. The importance of LAM as a marker reflects the fact that it is a major virulence factor in the infectious pathology of TB. Moreover, LAM is a loosely associated, but a large fractional component (˜40%) of the mycobacterial cell wall. LAM is therefore easily shed into the circulation system of an infected patient. Meta-analyses and other assessments have concluded that the tests for LAM in the serum and urine of infected patients by platforms that could potentially be used in the global fight against TB (i.e., conventional ELISA and lateral flow assays (LFA)) are, at best, of marginal value due to their poor clinical sensitivities and specificities.

The diagnostic strength of LAM as a serum marker for TB could be significantly improved by an assay approach with the ability to measure this marker in infected patient specimens at levels well below the reported limit of detection (LOD) of conventional ELISA (about 1 ng/mL, which is 10-100 times more sensitive than that of LFA). A sandwich immunoassay for the detection of LAM has been developed that combines gold nanoparticle (AuNP) labeling, anti-LAM monoclonal antibodies (mAbs), and readout by SERS. This approach exploits the strengths of SERS for the low-level quantification of biological analytes. This approach, which includes a novel sample pretreatment step, can reliably measure LAM in TB-positive patient sera at levels 100 times below those reported for the conventional ELISA test for this marker. The results of an assessment of the accuracy of this approach by analyzing sera from 24 TB-positive patients (culture-confirmed) and 10 healthy controls are presented.

Conventional ELISA procedures do not pretreat samples or pretreat samples using only heat and/or organic solvents such as methanol. Analysis of samples which underwent the novel pretreatments methods described herein by ELISA, however, showed a significant improvement in the limits of detection, such that much lower analyte concentrations could be detected. These results demonstrate the use of the disclosed methods as a tool for TB detection.

LAM is a major virulence factor in the infectious pathology of TB and has been found in serum and other body fluids (e.g., sputum, urine, and cerebral spinal fluid) of infected patients. LAM is one of the most heavily investigated antigenic markers for use in TB diagnostics. However, the conventional ELISA test routinely used as a standard for LAM testing can only detect this antigen in serum and other specimens down to a concentration of about 1 ng/mL, which has been shown in many cases to be inadequate for TB diagnosis.

Two factors which may impact the effectiveness of LAM as a serum marker for TB include: (1) the inherent limit of detection (LOD) of the conventional ELISA for LAM; and (2) the association of LAM with other serum components. Described herein is a novel sample pretreatment procedure that enables the measurement of LAM at an estimated LOD of 10 pg/mL as detected by SERS, which is approximately 100 times more sensitive than that reported for the conventional ELISA tests for this antigen. An assessment of the accuracy of this approach was performed using sera from 24 TB-positive patients (culture-confirmed) and 10 healthy controls. LAM was measurable in 21 of the 24 TB-positive specimens, but it was not detectable in any of the controls specimens. Notably, 17 of the TB-positive specimens contained LAM below the reported level detectable by the conventional ELISA test for this marker.

The novel pretreatment procedure also allows for the meaningful detection of LAM by ELISA, likely due to improved purification which more completely separates LAM from other serum components. The novel pretreatment methods described herein thus involve both of the factors discussed above regarding the use of LAM as a serum marker for TB. These results provide evidence of the clinical utility of LAM as a TB biomarker and also allow for multiple and varied assay systems to be used for its detection.

These methods may be extended for use in clinics and other point-of-care settings, along with applications to other disease markers, assay constructs, and other types of patient specimens. For example, the methods described herein are not only amenable to antibodies, including monoclonal and polyclonal antibodies, but also may be used in the detection and/or quantification of peptides, carbohydrates, lipids, antigens, DNA, RNA, genes or organic molecules, or any other type of analyte which may be used as an indicator of biological processes or responses to therapeutic intervention.

### SUMMARY

The present invention relates to methods for detecting lipoarabinomannan in a biological sample, comprising contacting the biological sample with an acid selected from the group consisting of perchloric acid, trifluoroacetic acid, and sulfosalicylic acid.

The present invention also provides for a method for diagnosing tuberculosis in a mammal, comprising contacting a serum sample of the mammal with an acid selected from the group consisting of perchloric acid, trifluoroacetic acid, and sulfosalicylic acid.

The present invention also provides for kits used to perform the methods described above. Other aspects of the invention will become apparent by consideration of the detailed description and accompanying drawings.

### BRIEF DESCRIPTION OF THE DRAWINGS

The drawings below are supplied in order to facilitate understanding of the Description and Examples provided herein.

FIG. 1 is a plot of protein content remaining in the supernatant of human serum samples after acid treatment as a function of acid molarity for trifluoroacetic acid, perchloric acid, and sulfosalicylic acid.

FIG. 2A and FIG. 2B show the SERS responses for various concentrations of galactomannan (GM) in pooled human serum. FIG. 2A compares the perchloric acid pretreatment of human serum before and after the addition of GM. FIG. 2B compares the EDTA-heat pretreatment of human serum before and after the addition of GM.

FIG. 3A-3C show a schematic illustration of the three main components of an exemplary SERS-based immunoassay approach for LAM: (FIG. 3A) ERL preparation; (FIG. 3B) capture substrate preparation; and (FIG. 3C) major assay steps.

FIG. 4A and FIG. 4B show a step-by-step schematic of an exemplary embodiment of the novel pretreatment procedure employing a decomplexation reagent to separate large molecules from proteins and other components in human serum. FIG. 4A shows steps 1, 2 and 3; FIG. 4B shows steps 4 and 5.

FIG. 5A and FIG. 5B show the result of an exemplary SERS-based immunoassay for LAM spiked into PBST.

FIG. 6A and FIG. 6B show the result of an exemplary SERS-based immunoassay for LAM spiked into untreated human serum.

FIG. 7A and FIG. 7B show the result of an exemplary SERS-based immunoassay for LAM spiked into pretreated human serum.

FIG. 8 shows the full dose-response plot from duplicate calibration runs for serum blanks (negative human serum after pretreatment) and for LAM spiked from 0.025 to 1000 ng/mL into negative human serum that was then pretreated before pipetting (20 μL) of the pretreated samples onto the capture substrates.

FIG. 9 shows the result of an exemplary SERS analysis of patient serum (pretreated) for the quantification of LAM represented in a bar chart.

FIG. 10 shows representative SERS spectra from patient serum (pretreated) samples.

FIG. 11A and FIG. 11B show dose-response curves for the results of an exemplary ELISA-based immunoassay detecting LAM in human serum with pretreatment (FIG. 11A) and without pretreatment (FIG. 11B).

FIG. 12 shows dose-response curves for the results of an exemplary SERS-based immunoassay detecting LAM in human serum without pretreatment and with pretreatment.

### DETAILED DESCRIPTION

Before any embodiments of the invention are explained in detail, it is to be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof, as well as additional items.

It also should be understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

It should be understood that, as used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 10%. In all cases, where the term “about” has been used to describe a value, it should be appreciated that this disclosure also supports the exact value.

Reference throughout this specification to “one embodiment,” “an embodiment,” or similar language means that a particular feature, structure, or characteristic described in connection with the embodiment is included in at least one embodiment of the invention provided herein. Thus, appearances of the phrases “in one embodiment,” “in an embodiment,” and similar language throughout this specification may, but do not necessarily, all refer to the same embodiment.

Furthermore, the described features, structures, or characteristics of the methods, compositions, and kits provided herein may be combined in any suitable manner in one or more embodiments. In the following description, numerous specific details are provided, to provide a thorough understanding of embodiments. One skilled in the relevant art will recognize, however, that the embodiments may be practiced without one or more of the specific details, or with other methods, components, materials, and so forth. In other instances, well-known structures, materials, or operations are not shown or described in detail to avoid obscuring aspects of the embodiments.

The methods disclosed herein demonstrate the potential of LAM to serve as a long sought-after antigenic marker for TB, particularly in view of the global needs and obstacles faced by TB diagnostics. In 2006, the Global Health Diagnostics Forum, convened by the Bill & Melinda Gates Foundation, estimated that a rapid and globally available diagnostic test for TB that has a clinical sensitivity ≧85% and a clinical specificity of ≧97% could help save ˜400,000 lives each year.

Clinical sensitivity (SN) and clinical specificity (SP) measure diagnostic test accuracy. SN is defined as the percentage of infected individuals correctly identified by the test as infected; it is expressed as: (TP/(TP+FN))100, where TP is the number of true positives and FN and is number of false negatives. SP is the percentage of uninfected subjects correctly identified by the tests as being uninfected; it is given as: (TN/(FP+TN))100, where TN is the number of true negatives and FP is the number of false positives (FP). A diagnostic test that is a perfect predictor of disease status has a SN of 100% and a SP of 100%.

The only platforms that currently meet both diagnostic metrics are microbial culturing and a NAAT test, but both are considered by the Forum to be too costly and complex for routine use in resource-limited settings. Cost and ease-of-use are pivotal in dictating the deployment of a test in regions of the world where it is needed the most. However, the most important diagnostic need for TB is the identification and validation of one or more antigens, either individually or in a panel, that can be used for the reliable and early diagnosis of the disease. The experiments described herein suggest that LAM, when combined with the strengths of SERS or ELISA detection and sample pretreatment, has the potential to meet these metrics, with a clinical sensitivity of up to 87.5% and clinical specificity of up to 100%.

The methods disclosed herein represent an emerging ultrasensitive detection motif for use in TB diagnostics, which is also extensible, thereby opening the possibility for the simultaneous detection of multiple TB markers as a means to enhance the sensitivity and specificity of the test.

Several development issues are involved with respect to the instrumentation, sample processing, and reagents used for the detection of analytes to become applicable beyond the research laboratory. For example, a system that can be hand held, battery powered, and requires minimal to no manual specimen manipulation is what is needed in the TB endemic regions. In addition, a low-cost-per-test diagnostics kit for TB, which incorporates stable regents (e.g., calibration standards, extrinsic Raman labels (ERLs), pre-made capture substrates, etc.) and materials for serum pretreatment, will need to be designed, packaged, and validated. Detection in such a manner may be feasible with the use of ELISA and/or SERS-based assays.

ELISA is a well-accepted diagnostic tool for detecting low levels of analytes. Similarly, SERS is a viable analytical diagnostic measurement tool. This relates, in large part, to the design of an assay in which the enhanced response for SERS is reproducibly managed. Reproducibility may be controlled, for example, by: (1) the size and shape distribution of the gold nanoparticles that constitute the ERL core; (2) the ability to form a monomolecular layer of Raman reporter molecules (RRMs) and mAbs on the ERLs; and (3) the use of a smooth gold capture substrate. The latter is relevant due to plasmonic coupling between the gold core of the ERL and the gold support of the capture substrate. UV-Vis spectrophotometry is used to maintain a fixed concentration of ERLs in the suspension used to tag the captured antigen. This integrated approach, which also includes tests to ensure consistency of reagents (e.g., mAb-Ag binding strength), can quantify serum constituents that may be of use as markers for the early diagnosis of diseases such as TB, with an accuracy and reproducibility that matches ELISA. Both the ELISA-and SERS-based detection technologies may be used for the detection of the TB marker LAM when coupled with the novel pretreatment methods described herein.

### EXAMPLES

Exemplary embodiments of the present disclosure are provided in the following examples. The examples are presented to illustrate the inventions disclosed herein and to assist one of ordinary skill in making and using the same. These are examples and not intended in any way to otherwise limit the scope of the inventions disclosed herein.

### Example 1

### Development of the Pretreatment Methods

A panel of reagents was evaluated for their ability to separate large molecules (15-65 kDa) from complexing proteins and other components in human serum as a soluble, as opposed to insoluble, product.

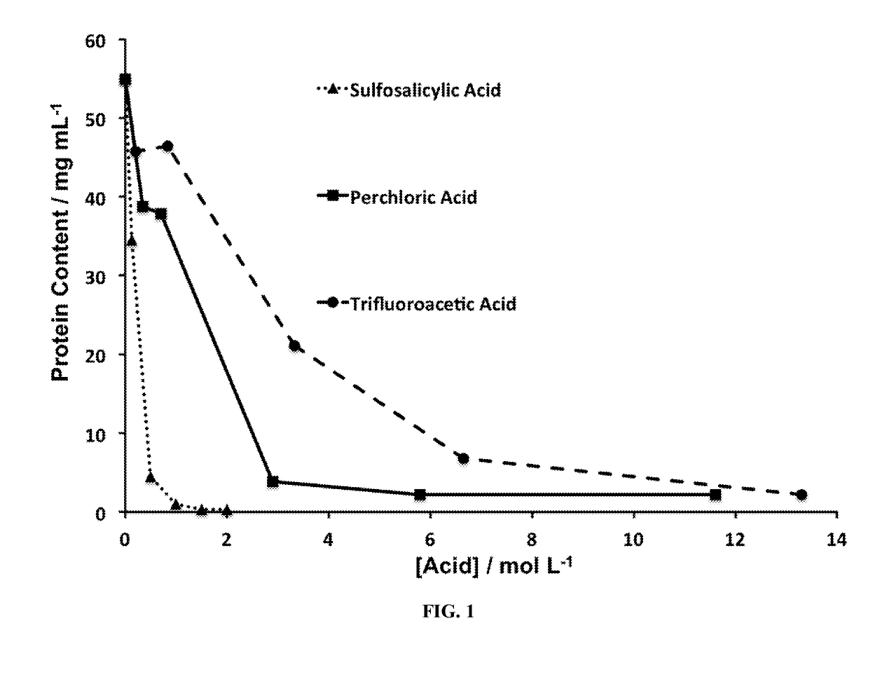

Separate aliquots (100 μL) of pooled human serum were treated with 10 μL aliquots of each of the reagents listed in Table 1. After addition, the treated serum samples were centrifuged at 12,045 g for 5 min. The pH of the supernatant was measured with a pH microelectrode. This measurement was followed by protein concentration measurements using UV/Vis spectroscopy (OD at 280 nm; bovine serum albumin standards used for calibration). Examples of the protein content found in the supernatant after treatment are plotted in FIG. 1 for the three acids tested as a function of acid molarity.

TABLE 1
pH and Protein Concentration in Human Serum Supernatant
after Treatment with Decomplexing Reagents.

| | Reagent | | Protein |
|---|---|---|---|
| | Concentration | pH of | Concentration |
| Reagent | (M) | Supernatant | (mg mL⁻¹) |
| Untreated Serum | | 7.70 | 54.96 |
| Perchloric Acid | 0.35 | 4.02 | 38.73 |
| | 0.70 | 2.83 | 37.87 |
| | 2.90 | 0.41 | 3.85 |
| | 5.80 | −0.02 | 2.17 |
| | 11.6 | −0.18 | 2.22 |
| Trifluoroacetic Acid | 0.21 | 4.16 | 45.71 |
| | 0.83 | 1.35 | 46.43 |
| | 3.33 | 0.47 | 21.09 |
| | 6.65 | 0.29 | 6.81 |
| | 13.3 | 0.01 | 2.20 |
| Sulfosalicylic Acid | 0.13 | 4.54 | 34.50 |
| | 0.50 | 2.46 | 4.51 |
| | 1.00 | 1.37 | 0.97 |
| | 1.50 | 0.98 | 0.33 |
| | 2.00 | 0.81 | 0.32 |
| Sodium | 0.87 | 9.05 | 41.99 |
| Hypochlorite | | | |
| (Bleach) | | | |
| 30% Hydrogen | 9.79 | 1.21 | 45.36 |
| Peroxide | | | |

FIG. 1 shows a plot of protein content remaining in the supernatant of human serum samples after acid treatment as a function of acid molarity for trifluoroacetic acid, perchloric acid, and sulfosalicylic acid. Perchloric acid was selected for additional testing with samples of LAM spiked into serum and with TB+ patient samples. Perchloric acid consistently yielded reproducible results and allowed for the detection of LAM in a variety of biological assays. The perchloric acid pretreatment procedure is rapid and minimizes any dilutions by employing highly concentrated reagents and does not require a two-step process, such as EDTA/heat followed by ethanol precipitation, for the detection of lipopolysaccharides.

### Example 2

### Comparative Example

In order to assess the capability for LAM in serum of the various strong acids (pKa <3), a standard calibration curve for LAM in human serum was determined, followed by pretreatment with each of the acids shown in Table 2. The same experimental procedures as described for Example 1 were used to collect the data presented here.

TABLE 2
pH and Protein Concentration in Human Serum Supernatant
after Treatment with Decomplexing Reagents.

| | Reagent | | Protein |
|---|---|---|---|
| | Concentration | pH of | Concentration |
| Reagent | (M) | Supernatant | (mg/mL) |
| Untreated Serum | | 7.70 | 54.96 |
| Perchloric Acid | 0.35 | 4.02 | 38.73 |
| | 0.70 | 2.83 | 37.87 |
| | 2.90 | 0.41 | 3.85 |
| | 5.80 | −0.02 | 2.17 |
| | 11.6 | −0.18 | 2.22 |
| Trifluoroacetic Acid | 0.21 | 4.16 | 45.71 |
| | 0.83 | 1.35 | 46.43 |
| | 3.33 | 0.47 | 21.09 |
| | 6.65 | 0.29 | 6.81 |
| | 13.3 | 0.01 | 2.20 |
| Sulfosalicylic Acid | 0.13 | 4.54 | 34.50 |
| | 0.50 | 2.46 | 4.51 |
| | 1.00 | 1.37 | 0.97 |
| | 1.50 | 0.98 | 0.33 |
| | 2.00 | 0.81 | 0.32 |
| Nitric Acid | 0.73 | 1.70 | 58.39 |
| | 1.45 | 0.49 | 42.70 |
| | 2.90 | -0.19 | 11.19 |
| | 5.80 | −0.41 | 8.87 |
| | 11.6 | −0.86 | 8.04 |
| Sulfuric Acid | 0.29 | 3.22 | 58.16 |
| | 0.58 | 1.59 | 59.70 |
| | 1.15 | 0.87 | 58.13 |
| | 4.60 | −0.20 | 43.47 |
| | 18.4 | −1.05 | 28.59 |
| Hydrochloric Acid | 0.38 | 4.14 | 59.12 |
| | 0.76 | 3.08 | 59.98 |
| | 1.51 | 0.92 | 58.31 |
| | 6.05 | −0.24 | 45.39 |
| | 12.1 | −0.41 | 35.46 |

Without being bound by theory, it is believed that the acid selectively aids in decomplexation of the LAM from serum proteins. The data in Table 2 shows that three strong acids (nitric acid, sulfuric acid and hydrochloric acid) are not as effective as perchloric acid, trifluoroacetic acid or sulfosalicylic acid at removing protein. Even sulfuric acid at 18.4 M, the strongest acid by molarity, is only capable of removing approximately half of the protein content during pretreatment of a 100 μL human serum sample. The most efficient reagents to free LAM from complexation and allow the LAM to partition into the solution phase in the pretreatment, are perchloric acid, trifluoroacetic acid, and sulfosalicylic acid.

The samples were then analyzed by SERS to determine if there was a correlation between protein concentrations in the supernatant and detection of LAM. The SERS response for 0.5 ng/mL of LAM, normalized to perchloric acid pretreatment as producing a signal of 100%, is shown in Table 3. The SERS response for the various pretreatments was obtained following the procedures described in Example 4.

TABLE 3
Comparison of SERS immunoassay response detecting LAM
as a function of pretreatment acid, normalized to the perchloric
acid pretreatment response as a percentage.

| | SERS Response for 0.5 ng/mL of LAM |
|---|---|
| Pretreatment Acid | (Normalized to PCA in %) |
| Perchloric acid (PCA) | 100 ± 4 |
| Trifluoroacetic Acid | 110 ± 9 |
| Sulfosalicylic Acid | 86 ± 5 |
| Nitric Acid | 38 ± 3 |
| Sulfuric Acid | 39 ± 2 |
| Hydrochloric Acid | 50 ± 5 |

As was the case for levels of protein, the assay responses from the nitric acid, sulfuric acid, and hydrochloric acid pretreated samples are less than half of the obtained response from perchloric acid pretreatment. This data indicates that only a select few acids have the potential to effectively liberate LAM from complexation and remove unwanted species from the serum sample, allowing for detection and quantification of the LAM in a biological assay. This data shows that LAM is unique and requires a specific pretreatment regimen to be effectively decomplexed for detection.

### Example 3

### Comparative Example

Galactomannan (GM) is a LAM-like antigenic marker used in the detection of invasive aspergillosis. The novel pretreatment methods developed for LAM were evaluated for use in the pretreatment of GM, a polysaccharide.

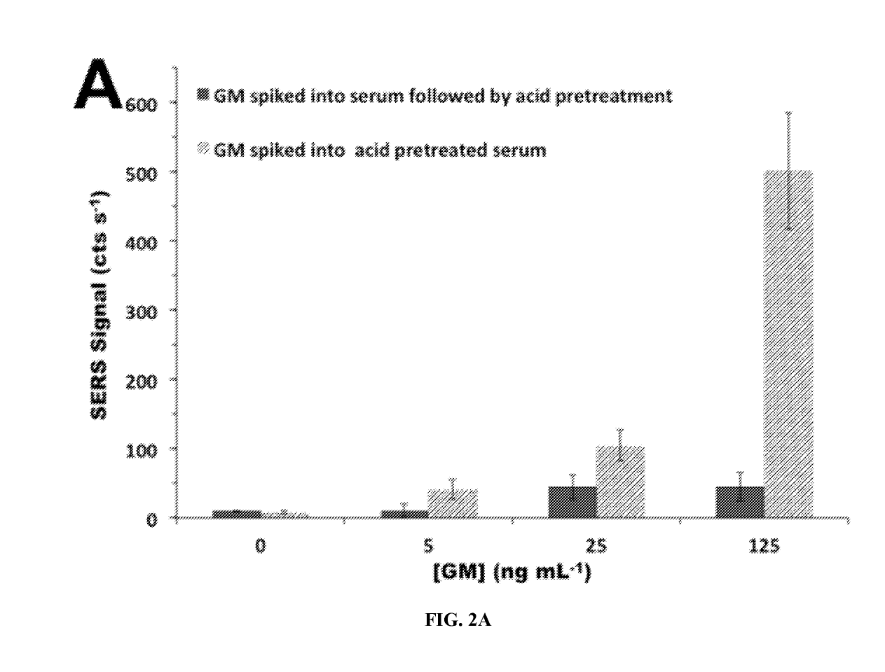

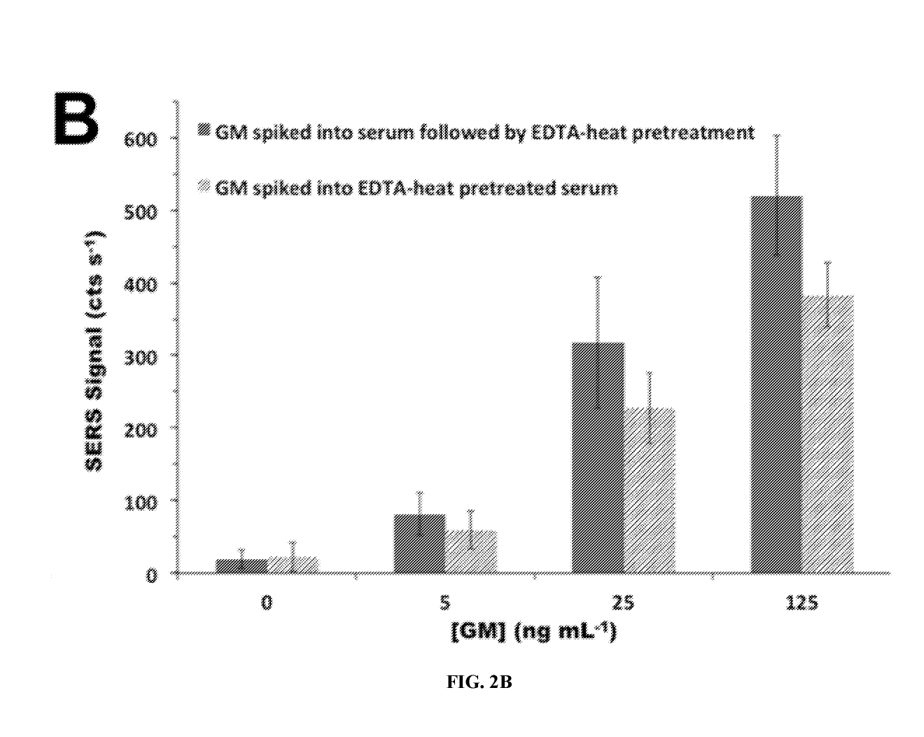

The SERS response for these pretreatment experiments was obtained following the procedures described in Example 4. FIG. 2 shows SERS responses as a function of the GM concentration that was spiked into serum before and after each of the pretreatment protocols.

FIG. 2A shows the SERS responses for various concentrations of galactomannan (GM) in pooled human serum, comparing a perchloric acid pretreatment of human serum before and after the addition of the GM spike. FIG. 2B shows the SERS responses for various concentrations of galactomannan (GM) in pooled human serum, comparing an EDTA-heat pretreatment of human serum before and after the addition of the GM spike.

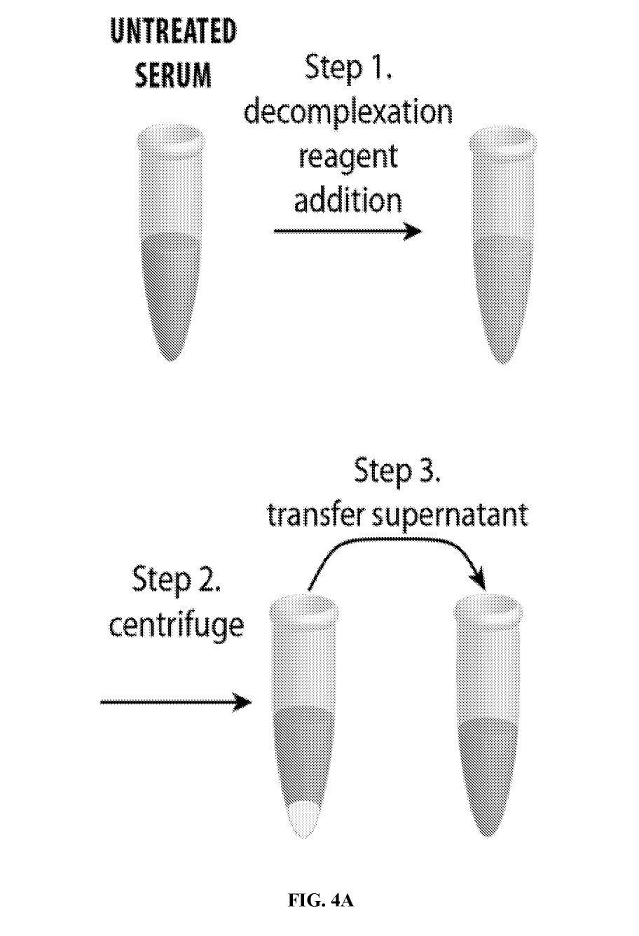

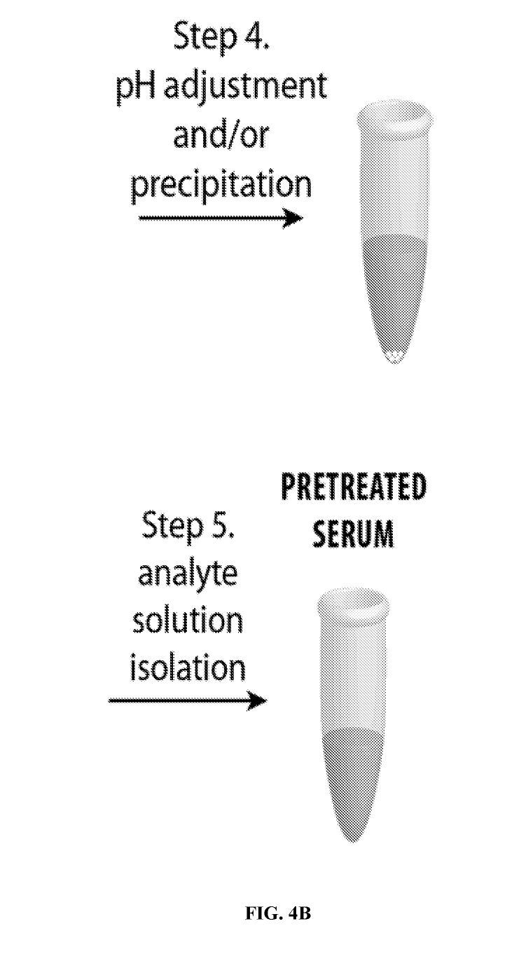

The procedure for the perchloric acid pretreatment for GM in human serum was similar to the procedure outlined in FIG. 4. The pretreatment is either carried out before or after the spiking of GM. A 0.2 mL human serum sample was contacted with 6 μL of HClO₄ (70%, Sigma-Aldrich) to lower the pH to ˜2 and formed a milky suspension. After vortexing for 10 seconds and centrifuging at 8000 g for 10 min, 150 μL of the resulting supernatant was transferred to a second centrifuge tube and neutralized to pH 7.5 with an aqueous solution of KOH (2.0 M). Similarly, the EDTA-heat pretreatment procedure is either carried out before or after the spiking of GM. It uses a 300 μL serum sample that is mixed with 100 μL of 4% EDTA and heated to 95° C. for 30 min. This solution is then centrifuged at 8000 g for 10 min, and 225 μL of the supernatant was removed for analysis. The remainder of the procedure for the two pretreatment protocols, including the SERS-based detection using the pretreated solutions, was carried out analogously to the procedure described for Example 4 with the exception of using a mAB specific for GM (WF-AF-1) instead of the mAB for LAM.

In contrast to the pretreatment of LAM in serum, perchloric acid pretreatment exhibited a negative effect in the detection of GM by SERS immunoassay (FIG. 2A). It appeared that perchloric acid degrades GM. With a different pretreatment method, one involving the addition of EDTA in conjunction with heating to 95° C. for 30 min, GM was detected (FIG. 2B).

### Example 4

### Use of the Pretreatment Methods for the Detection of LAM with a SERS-Based Immunoassay.

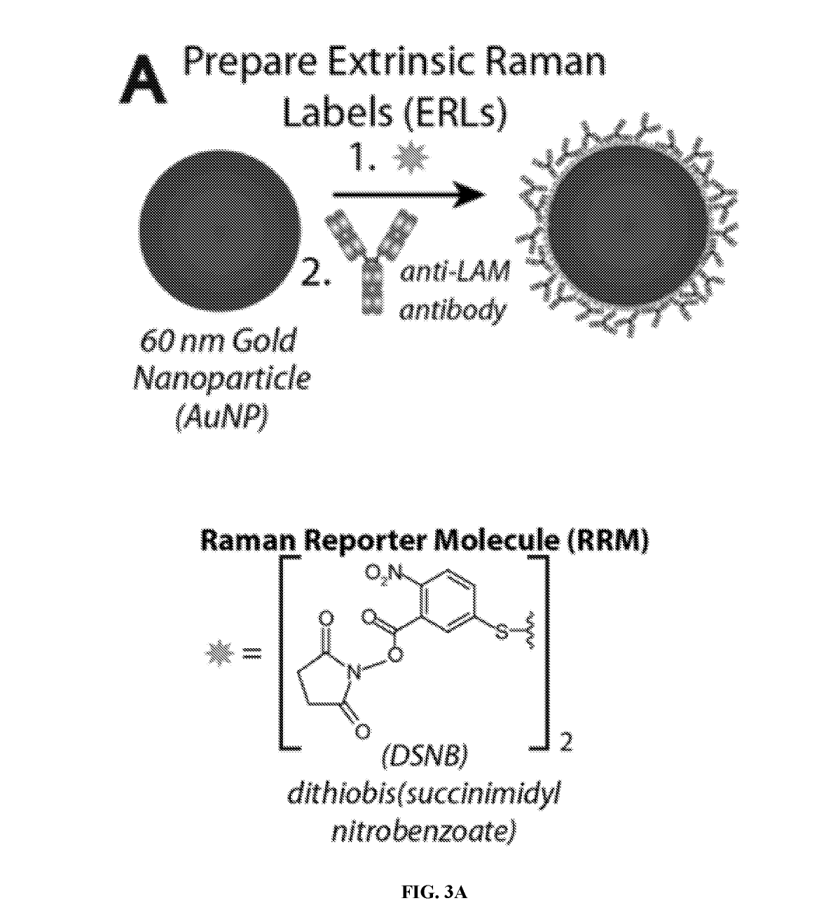

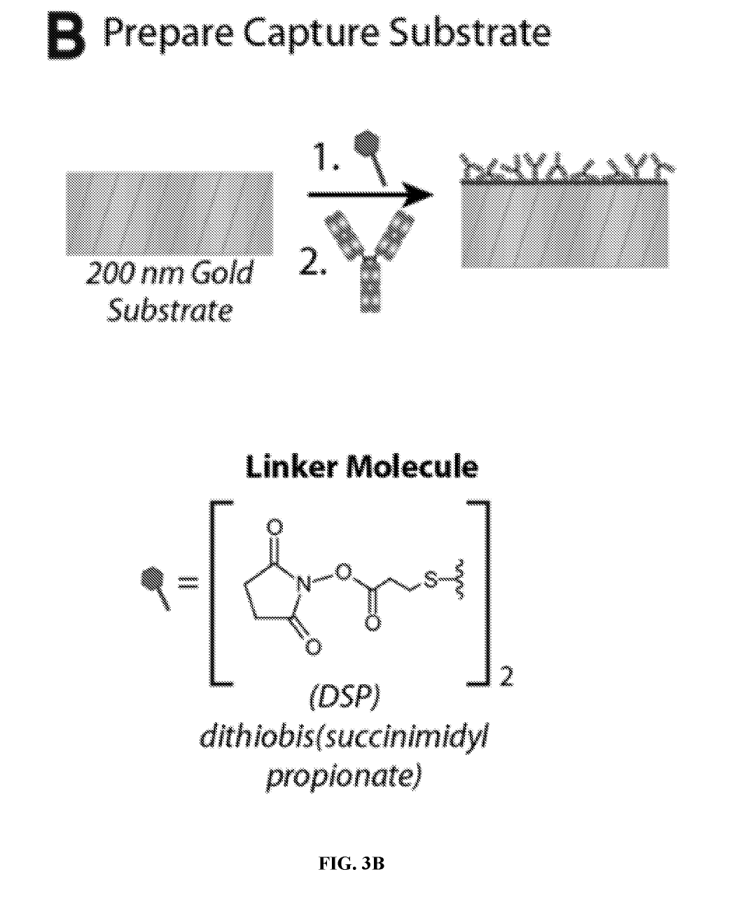

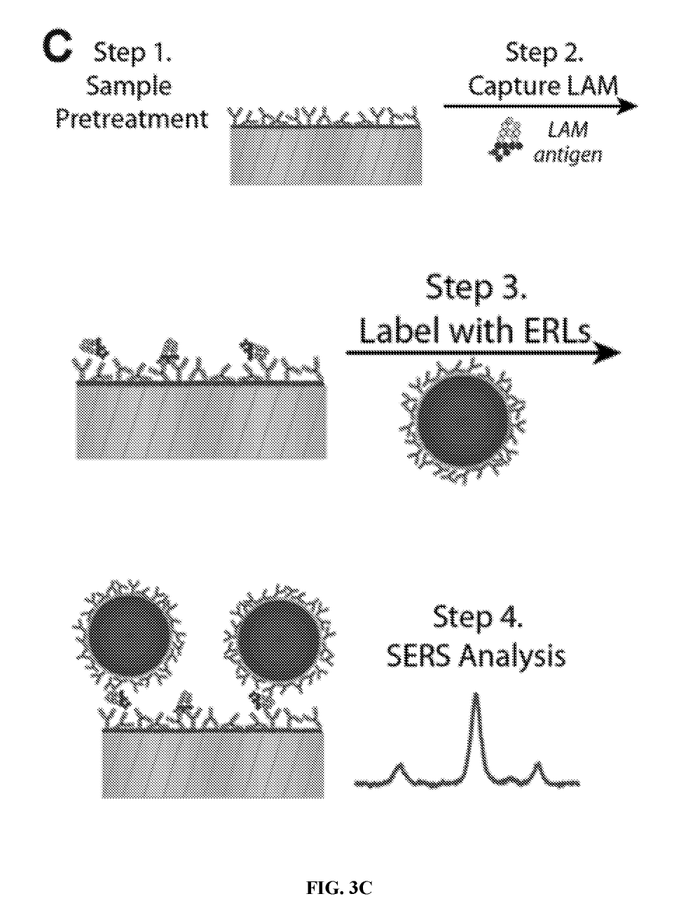

Assay Format. FIGS. 3A-3C illustrate an embodiment of a SERS-based immunoassay sandwiching LAM between an extrinsic Raman label (ERL) and capture substrate. The first two procedures (FIG. 3A and 3B) are completed prior to the actual assay. The assay (FIG. 3C) is carried out by incubating a pretreated serum sample (20 μL) for 1 hour at room temperature with the capture substrate. The samples are then rinsed, exposed to ERLs (20 μL, overnight for convenience, 16 h), rinsed again, dried under ambient conditions, and analyzed by SERS.

ERLs are prepared by modifying 60-nm AuNPs with a thiolate monolayer that was formed by the spontaneous adsorption of the disulfide-bearing Raman reporter molecule (RRM) 5-5′-dithiobis(succinimidyl-2-nitrobenzoate) (DSNB), as shown in FIG. 3A. This step was followed by the immobilization of a layer of anti-LAM mAbs via an amidization reaction between the succinimidyl group of the RRM and amine residues at the mAb periphery. This construction places the Raman scattering centers of the RRM monolayer (e.g., its symmetric nitro stretch, vs(NO₂)) in close proximity to the AuNP surface in order to maximize the SERS signal. The smooth, glass-supported gold (˜200 nm thick) capture substrate was also coated with anti-LAM mAbs via a monolayer formed with dithiobis(succinimidyl propionate) (DSP), as shown in FIG. 3B. As a result, the presence of captured LAM in a sample was indirectly signaled by the characteristic Raman spectrum of the RRM and the amount of LAM is indirectly quantified by the strength of the most intense spectral feature.

Extrinsic Raman Labels (ERLs). The preparation and plasmonic signal optimization of ERLs have been described and are summarized in FIG. 3A. First, an aqueous suspension of 60-nm (nominal diameter) AuNPs (NanoPartz, Loveland, Colo.) in 2.0 mM borate buffer (BB, pH 8.5, Fisher Scientific) was mixed for 1.5 h with DSNB (10.0 mM in acetonitrile, spectroscopy grade, Sigma-Aldrich) at 4° C. This step yielded a DSNB-derived thiolate monolayer on the AuNP surface that forms via disulfide cleavage. Next, 10 μL (100 μL) of the CS906.7 anti-LAM mAb (Colorado State University) were added using a recently calibrated pipette (Pipette Repair Service, Midlothian, Va.) into the AuNP suspension and incubated for 1 h, which immobilized the mAbs to the AuNP surface at 4° C. This step was followed by the addition of a 100-μL aliquot of 10% (w/v) bovine serum albumin (BSA, Sigma-Aldrich) solution in 2.0 mM BB to block unreacted succinimidyl groups and to stabilize the colloidal suspension; this process was carried out at room temperature under continuous agitation for 1 h. The suspension was then centrifuged at ˜2,000 g for 10 min, and the clear supernatant carefully removed. The ERL pellet was resuspended in 1.0 mL of 1% BSA in 2.0 mM BB. Centrifugation and resuspension were repeated two more times, with the final resuspension using 0.5 mL of 2% BSA in 2.0 mM BB and 150 mM NaCl (Fisher Scientific) to achieve an ERL concentration 4.0×1010 particles/mL. The ERL concentration was verified by the spectrophotometric method described by Haiss and colleagues. Measurements of the amounts of DSNB and mAbs coated on the ERLS varied by only ±5.0 and ±10.2%, respectively.

Capture Substrate. Capture substrates (FIG. 3B) were prepared with 1×1-cm glass squares that supported a 200-nm layer of template stripped gold (TSG). A 2-mm diameter address was created in the center of the substrate by octadecanethiol (ODT, Fluka) microprinting with polydimethylsiloxane (PDMS, Dow Corning SlyGuard). The ODT layer produced a hydrophobic boundary around an uncoated 2-mm address, which was then modified for 1 h with an ethanolic solution of DSP (0.1 mM, Fisher Scientific). Next, the DSP monolayer was reacted with a 20.0-μL drop of capture antibody (2.5 μg/mL, CS906.7 anti-LAM mAbs) for 1 h to tether anti-LAM mAbs via amide linkages. Next, the substrate was rinsed three times with phosphate buffered saline containing 0.1% Tween 20 (PBST, pH 7.4, Fisher Scientific); blocked with 20 μL of StartingBlock® (Thermo Scientific) for 1 h; rinsed three more times with PBST; and exposed (FIG. 3C) to 20.0 μL of a LAM-containing sample After 1 hour, the samples were rinsed three times with 2.0 mM BB (150 mM NaCl and 0.1% Tween 20), exposed to 20.0 μL of the ERL suspension, and incubated overnight. Finally, the samples were rinsed with 2.0 mM BB, containing 10.0 mM NaCl and 0.10% Tween 20, and dried under ambient conditions for ˜1 hour prior to SERS interrogation. Optical ellipsometry measurements indicated that the thicknesses of the capture coating varied by ±12.2%.

Instrumentation, Antigen Capture/Labeling and Readout, and Data Analysis. The Raman instrument used for data collection was a modified NanoRaman I system. This instrument has three primary components: laser excitation source, fiber optic probe, and spectrograph. The light source is a 22-mW, 632.8-nm HeNe laser with a spectrograph consisting of an f/2.0 Czerny Tuner imaging spectrometer with 6-8 cm⁻¹ resolution and a Kodak 0401E charged coupled device (CCD) thermoelectrically cooled to 0° C.

SERS readout was performed after the samples had fully dried under ambient conditions (about 1 hour). Raman spectra were collected by irradiating a 20-μm spot on the sample surface at 3.0 mW of laser power and a 1-s integration time. The laser power was checked periodically in each run and varied by 0.1 mW at most. Each sample was analyzed at 10 different substrate locations with duplicates of each calibrant concentration. The sera used for the development of the assay and the generation of calibration curves (i.e., serum spiked with LAM) was Human AB Serum (Mediatech, Inc., Manassas, Va.). This product, referred to hereafter as negative human serum, was prepared by pooling and sterilizing donor plasma collected at centers across the U.S. These samples were slowly thawed in the laboratory to room temperature after being stored at −30° C. Due to the small volumes received for the TB-positive and TB-negative serum specimens (approximately 100 μL), the patient serum samples were run only as duplicates. As a consequence, the levels of LAM in all patient samples are reported as averages and uncertainties as the range of the values from reading two separate substrates prepared from a single specimen. All spectra were baseline corrected and the height of the symmetric nitro stretch, vs(NO₂), at 1336 cm⁻¹ of the RRM was used for quantification. All calibration data are presented as the average and standard deviation of the collected spectra (20 spectra from 10 different locations per sample) in which all preparations for each substrate were independent of each other. The LOD was defined as the signal from a point on the calibration curve that matched the blank signal plus three times its standard deviation.

Monoclonal Antibody Selection. Three IgG₃ subclass, LAM-binding mAbs (anti-LAM mAbs) were screened for effectiveness for use with the SERS assay (FIG. 3). These mAbs, designated as CS906.1, CS906.7, and CS907.41 were first prepared and characterized for reactivity at Colorado State University in 1987, and were tested in each of their possible nine combinations for antigen capture and/or labeling of the captured antigen by measuring the SERS response of PBST spiked at 5.0 μg/ml of LAM. The levels of nonspecific ERL adsorption were also determined for blank PBST. These results indicate that the signal using CS906.7 for both the capture and labeling of LAM was about two times stronger than any of the other eight combinations. In contrast, all nine blank responses differed by amounts barely distinguishable by statistical analysis. Based on these results, CS906.7 was used as the capture and labeling mAb in all subsequent experiments. The epitope structures of LAM that are recognized by CS906.7 have not been characterized, however, the structure of LAM is consistent with the presence of a multiplicity of antigenic determinants with structural similarities that could react with CS906.7.

Serum Pretreatment. The direct detection of LAM spiked into serum without sample pretreatment yielded signal strengths well below those for LAM spiked into PBST, which was suspected to be a consequence of immunocomplex formation between LAM and various constituents in serum. A series of experiments were therefore designed to test various reagents to identify a means to disrupt the immunocomplexes. As a result of these experiments, a pretreatment procedure was developed to induce the disruption of LAM immunocomplexes, putatively via protein decomplexation.

This procedure has five steps, as outlined in FIGS. 4A and 4B. It begins (Step 1) by adding 2.0 μL of HClO₄ (70%, Sigma-Aldrich) to 100.0 μL of each calibration/patient sample in a small centrifuge tube, which brings the pH to ˜2 and forms a milky suspension. After vortexing for 10 seconds and centrifuging at 13,000 g for 5 min (Step 2), 75 μL of the resulting supernatant was transferred to a second centrifuge tube (Step 3) and neutralized to pH 7.5 with 6.0 μL of an aqueous solution of K₂CO₃ (2.0 M, Fisher Scientific) (Step 4). The samples were then cooled to 4° C. for 30 min and allowed to warm to room temperature (about 20 min) before being pipetted (20 μL) onto the capture substrate (Step 5).

Patient Specimens. All patient specimen experiments and healthy control collections were performed under approved IRB protocols at the University of Utah and Colorado State University in a biosafety cabinet contained in a BSL-2 (enhanced) laboratory.

The TB-positive sera were collected from patients enrolled in the Tuberculosis Trials Consortium Study Group 22 (TBTC-22) with culture-confirmed cavitary TB. This study group participated in a randomized clinical trial that was designed to test the effectiveness of the anti-TB drugs rifapentine and isoniazid in treating pulmonary tuberculosis in adult, HIV-negative patients. The de-identified samples were procured by Colorado State University from the Centers for Disease Control and Prevention (CDC) after TBTC-22 approval. This specimen set consisted of 24 different serum samples, each at a volume of ˜100 μL. No information with regard to treatment status (e.g., drug regimen or time course of treatment) for any of these specimens was available. However, tests for immunoblot reactivity confirmed the presence of anti-LAM antibodies in all TB-positive specimens, but not in any of the healthy controls (data not shown), which suggests that there was a high likelihood that LAM would also be present in the TBTC-22 study serum specimens.

Healthy, non-endemic control sera, referred to hereafter as healthy controls, were obtained from U.S.-born residents of Colorado. These non-Bacillus Calmette—Guérin (BCG)-vaccinated residents gave informed consent to participate in a study of reactivity to M leprae and Mtb antigens. These residents had no known exposure to TB or leprosy and did not work in a mycobacterial laboratory.

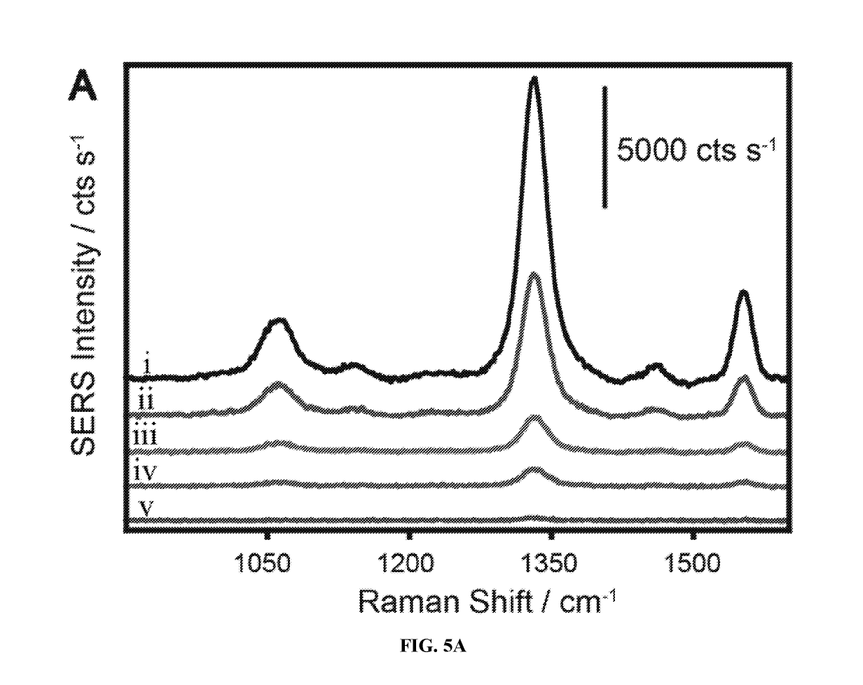

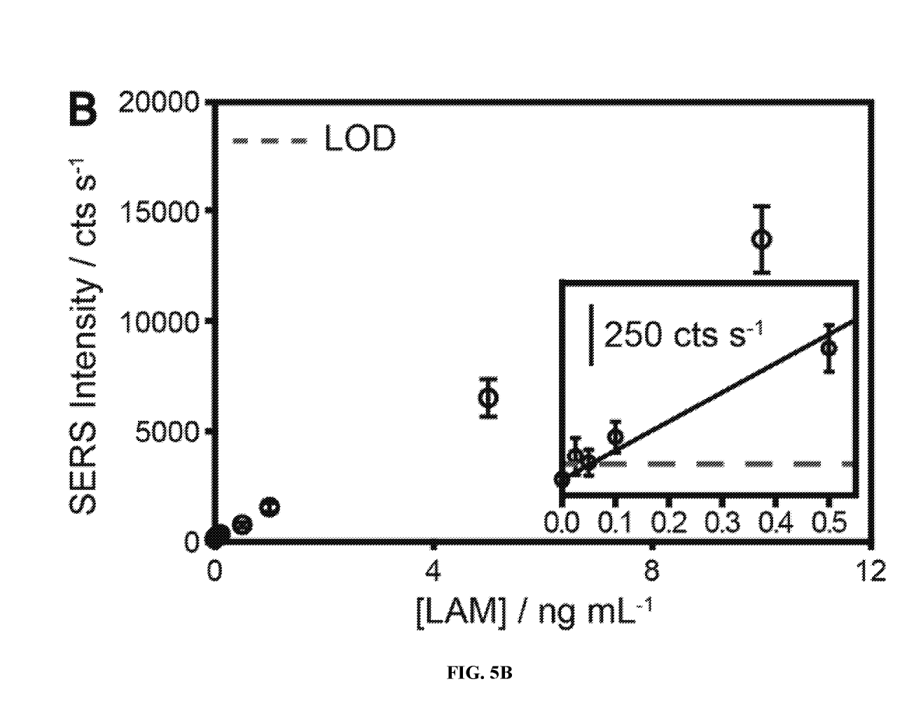

LAM Spiked into PBST. A set of experiments were designed and carried out to gauge the potential performance of the assay (FIG. 3) by spiking LAM at different amounts in a simple matrix like PBST. The SERS spectra and dose-response plot are presented in FIGS. 5A and 5B, respectively, and include measurements on PBST blanks (PBST devoid of LAM) and of LAM spiked into PBST at levels up to about 10 ng/mL.

FIG. 5A shows the SERS spectra for calibration using LAM-spiked PBST: (i) 10; (ii) 5.0; (iii) 1.0; (iv) 0.5; and (v) 0.0 ng/mL. The spectra are offset vertically for visualization. FIG. 5B shows the dose-response plot from the average of duplicate calibration runs (20 μL) for LAM spiked into PBST at differing levels (0.025 to 10 ng/mL) and for blank PBST. The LOD was calculated to be ˜50 pg/mL (˜3 pM). The inset in FIG. 5B plots the calibration data for the PBST blank and for LAM spiked into PBST at levels from 0.025 to 0.500 ng/mL. The equation for the linear fit to the data is (y=1296×+123; R²>0.98). The signal at the cutoff for the LOD is indicated by the dashed line in the inset.

The SERS spectra are shown in FIG. 5A. There are three important points to draw from these spectra. First, all of the observable spectral features can be assigned to functional groups of the RRM monolayer on the ERLs (e.g., vs(NO₂) at 1336 cm⁻¹ and aromatic ring mode at 1558 cm⁻¹ of the DSNB-derived coating). None of the vibrational modes of the anti-LAM mAb coating on the ERLs are detectably enhanced. Second, the strengths of the spectral features increase with increasing amounts of LAM. This dependence follows expectations for a sandwich immunoassay. Third, there is evidence for a small, but measureable level of ERL adsorption in the spectrum for the PBST blanks. This observation was attributed, at least in part, to the effectiveness of the blocking agent and other reagent preparative procedures to reduce nonspecific adsorption.

The dose-response plot is shown in FIG. 5B. It was constructed from the average signal for the strongest feature in the SERS spectrum, vs(NO₂), from 10 different locations per sample from duplicate calibration runs. The response at low levels of LAM follows a linear dependence. Though not shown, the response at higher amounts of LAM approaches a limiting value as mAb binding sites on the capture substrate begin to saturate. The LOD, defined by the response on the calibration plot that matches the blank signal plus three times its standard deviation, is calculated to be ˜50 pg/mL, a value ˜20 times below that for LAM by conventional ELISA.

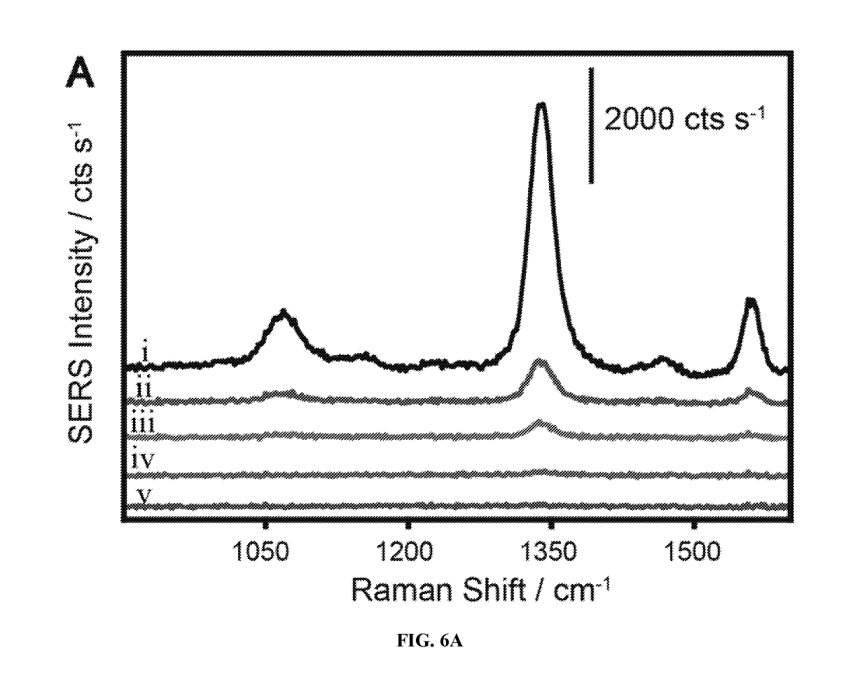

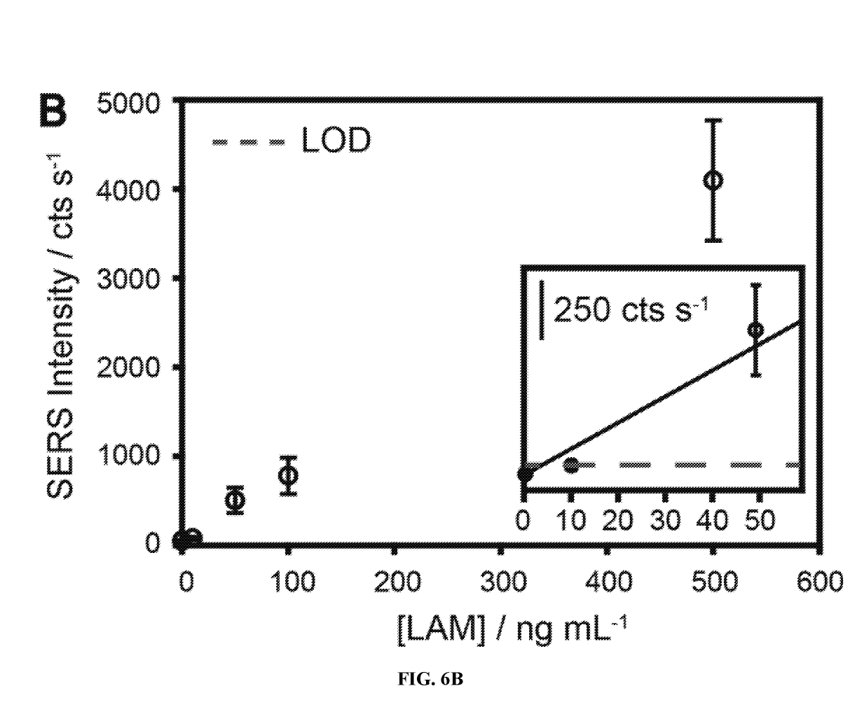

LAM Spiked into Untreated Human Serum. The samples for these experiments were prepared by spiking LAM into negative human serum. These samples were then briefly vortexed for mixing. The next steps followed the same capture and labeling procedures used for the PBST samples, including pipetting the spiked serum samples directly onto capture substrate. The SERS spectra and dose-response plot that resulted are shown in FIGS. 6A and 6B, respectively.

FIG. 6A shows the SERS spectra from a calibration run using LAM-spiked negative human serum: (i) 500 (ii) 100; (iii) 50; (iv) 10; and (v) 0.0 ng/mL. FIG. 6B shows the dose-response plot from averaging duplicate calibration runs (20 μL) for LAM spiked from 10 to 500 ng/mL and a negative (untreated serum) control sample. The LOD was estimated to be ˜4 ng/mL (0.24 nM). It was determined as the signal on the calibration plot that matches the blank signal plus three times its standard deviation via the data shown in the inset (y=8×+50; R²>0.99). The spectra are offset vertically for visualization. The signal at the cutoff for the LOD is indicated by the dashed line in the inset.

The strength of the responses for LAM spiked into negative human serum are much weaker than those for LAM spiked into PBST. For example, the response for LAM spiked into negative human serum at 5.0 ng/mL is just over 700 cts/s, which is close to that of the response for LAM spiked into PBST at 0.5 ng/mL. The amount of nonspecific ERL adsorption, however, is slightly lower, about two times as judged by the y-intercepts of the linear fits to the data given in the insets of FIGS. 5B and 6B. These two measurement metrics combine to yield a LOD for LAM in serum (untreated) of about 4 ng/mL, which is approximately 80 times less sensitive than that in PBST.

LAM Spiked into Pretreated Human Serum. The degradation of the LOD for LAM spiked into human serum lead to speculation that the assay using untreated serum was negatively affected by the formation of immunocomplexes of LAM with proteins and possibly other serum constituents. Indeed, there is a growing body of evidence for the presence of immunocomplexes for LAM in human serum, the most recent being the strong association of LAM with high density lipoproteins (HDLs). Several different methods were systematically evaluated as a means to disrupt possible immunocomplexes formed between LAM and serum constituents.

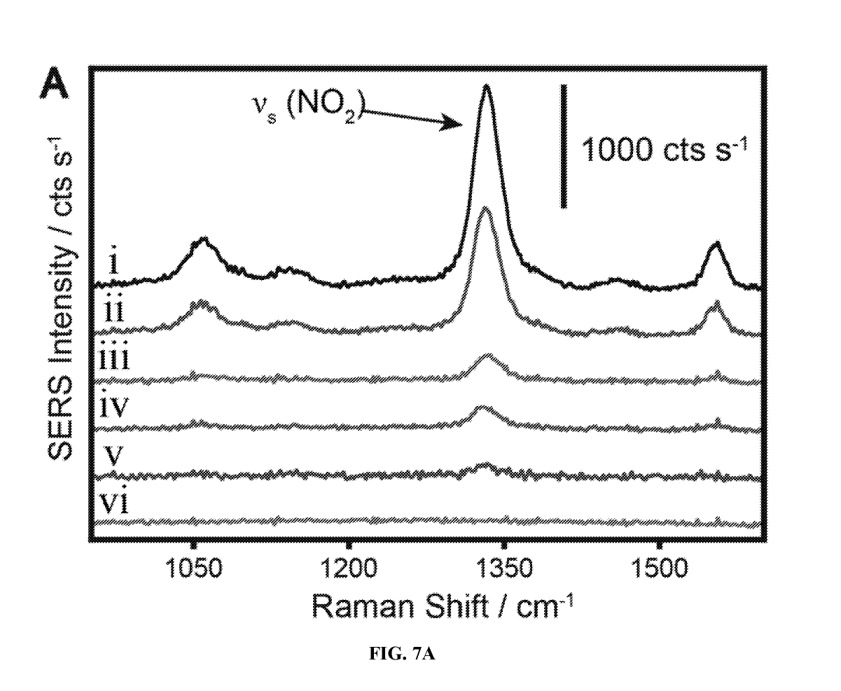

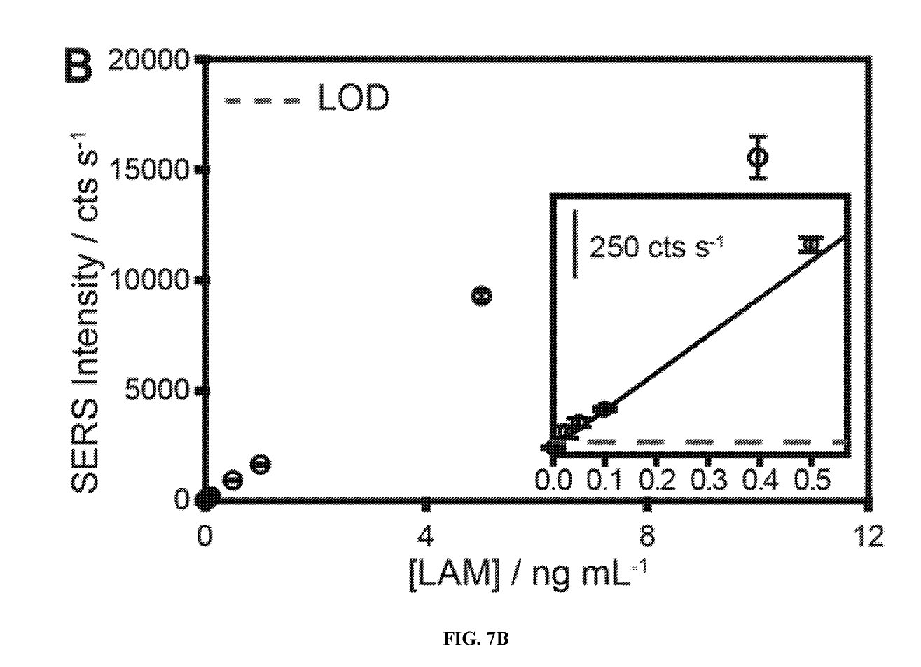

The first experiment tested a simple heat-based pretreatment for human serum (about 90° C. for 5 min, followed by centrifugation and supernatant collection) based on work performed and used in the past for LAM and for other assays in which the possible impact of immunocomplexes was of concern. Pretreating LAM spiked into serum by this procedure, however, proved to be only marginally useful. The utility of various decomplexation methods, including acidification, was also investigated. As is apparent from the data in FIGS. 7A and 7B, the acidification of serum with HClO₄ to a pH of about 2, and subsequent neutralization with K₂CO₃ proved to be effective. This pretreatment approach reduced the protein level in the supernatant to less than ˜4% of that in serum before pretreatment, as judged from the spectrophotometrically determined changes in absorbance at 280 nm. The other pretreatment reagents were not as effective in reducing the serum levels in the resulting supernatant, with only incremental improvements over the determination of LAM in untreated serum that is shown in FIGS. 6A and 6B.

FIG. 7A shows the SERS spectra using LAM-spiked negative pretreated human serum: (i) 1.0 (ii) 0.50; (iii) 0.10; (iv) 0.05; (v) 0.025; and (vi) 0.0 ng/mL. FIG. 7B shows the dose-response plot for duplicate calibration runs (20 μL, pretreated serum samples) for LAM spiked from 0.025 to 10 ng/mL and a negative (pretreated serum) control sample. The LOD was estimated to be ˜10 pg/mL (˜1 pM). It was determined as the signal on the calibration plot that matches the blank signal plus three times its standard deviation via the data shown in the inset (y=1665×+30; R²>0.99). The spectra are offset vertically for visualization. The signal at the cutoff for the LOD is indicated by the dashed line in the inset.

The results from using the perchloric acid pretreatment method on LAM that was spiked (0-1 ng/mL) into negative human sera are shown in FIG. 7A. The strengths of the SERS responses have not only returned to the levels for LAM spiked into PBST observed in FIG. 5A, but are actually slightly stronger. The origin of the high analytical sensitivity for the LAM assay in pretreated serum with respect to that in PBST may be related to the difference in the serum specimen after pretreatment (pH ˜9) and that of PBST (pH 7.4, with 0.1% Tween 20).

The responses for the pretreated serum blanks are slightly lower than those of the PBST blanks. Pretreatment therefore provides at least two positive attributes. It markedly improves the ability to detect LAM spiked into negative human serum while also reducing the level of observable nonspecific ERL adsorption.

The dose-response plot from duplicate calibration runs for LAM spiked into negative human serum and pretreated as described above is shown in FIG. 7B. This plot represents the average signal from 10 different locations on each sample. Like the data for LAM spiked into PBST (FIG. 5B), the response at low LAM levels again follows a linear dependence. Notably, the estimated LOD is approximately 10 pg/mL, which is roughly two orders of magnitude more sensitive than that reported for conventional ELISA in the analysis of LAM in human sera and in other matrices common in TB diagnostics (i.e., urine, cerebral spinal fluid, and sputum).

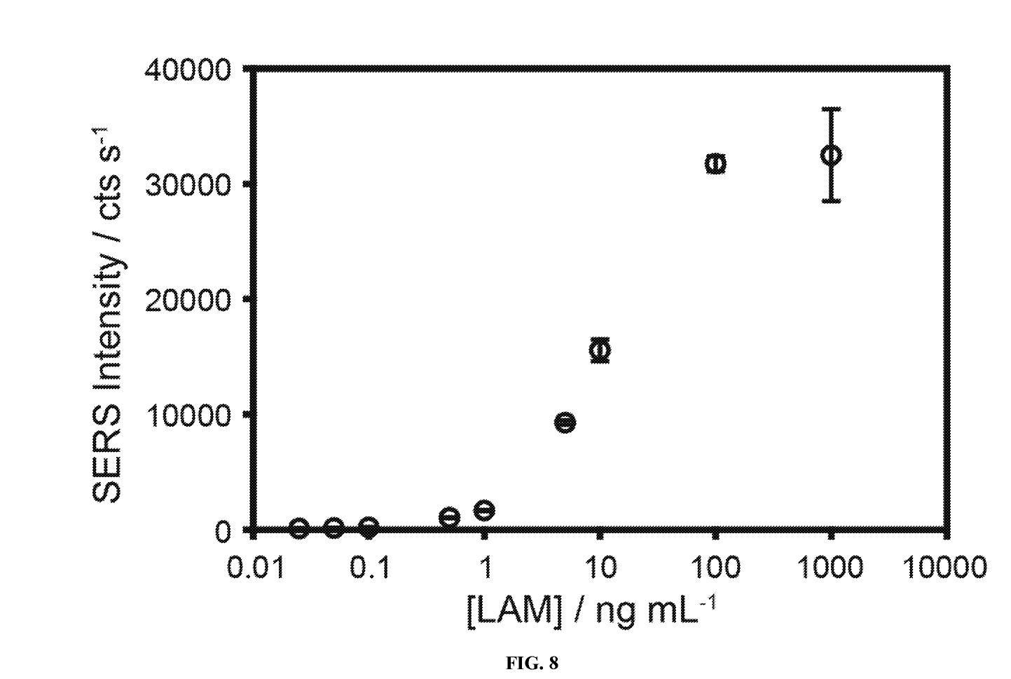

The response at low LAM levels also plateaus at higher amounts of LAM as mAb binding sites on the capture substrate begin to saturate, as shown in FIG. 8. FIG. 8 shows the full dose-response plot from duplicate calibration runs for serum blanks (negative human serum after pretreatment) and for LAM spiked from 0.025 to 1000 ng/mL into negative human serum that was then pretreated before pipetting (20 μL) of the pretreated samples onto the capture substrates. A summary of the SERS responses and the corresponding LAM concentrations for all 34 specimens are given in Table 4.

TABLE 4
Patient samples with SERS responses and
calculated LAM concentrations.

| Sample ID | SERS/cts s⁻¹ | [LAM]/ng mL⁻¹ |
|---|---|---|
| 1 | 728 ± 41 | 0.42 ± 0.02 |
| 2 | 282 ± 61 | 0.15 ± 0.04 |
| 3 | 219 ± 16 | 0.11 ± 0.01 |
| 4 | 2001 ± 170 | 1.18 ± 0.10 |
| 5 | 43 ± 3 | 0.01 ± 0.00 |
| 6 | 301 ± 86 | 0.16 ± 0.05 |
| 7 | 131 ± 67 | 0.06 ± 0.04 |
| 8 | 1059 ± 194 | 0.62 ± 0.12 |
| 9 | 1034 ± 116 | 0.60 ± 0.07 |
| 10 | 533 ± 96 | 0.30 ± 0.06 |
| 11 | 3710 ± 208 | 2.21 ± 0.12 |
| 12 | 1728 ± 61 | 1.02 ± 0.04 |
| 13 | 295 ± 31 | 0.16 ± 0.02 |
| 14 | 1065 ± 61 | 0.62 ± 0.04 |
| 15 | 37 ± 9 | 0.00 ± 0.01 |
| 16 | 1096 ± 214 | 0.64 ± 0.13 |
| 17 | 3349 ± 173 | 1.99 ± 0.10 |
| 18 | 1460 ± 36 | 0.86 ± 0.02 |
| 19 | 187 ± 60 | 0.09 ± 0.04 |
| 20 | 803 ± 139 | 0.46 ± 0.08 |
| 21 | 814 ± 129 | 0.47 ± 0.08 |
| 22 | 553 ± 208 | 0.31 ± 0.12 |
| 23 | 356 ± 115 | 0.20 ± 0.07 |
| 24 | 509 ± 146 | 0.29 ± 0.09 |
| 25 | 36 ± 4 | 0.00 ± 0.00 |
| 26 | 60 ± 15 | 0.02 ± 0.01 |
| 27 | 47 ± 11 | 0.01 ± 0.01 |
| 28 | 42 ± 14 | 0.01 ± 0.01 |
| 29 | 44 ± 7 | 0.01 ± 0.00 |
| 30 | 59 ± 27 | 0.02 ± 0.02 |
| 31 | 60 ± 21 | 0.02 ± 0.01 |
| 32 | 35 ± 8 | 0.00 ± 0.00 |
| 33 | 41 ± 27 | 0.01 ± 0.02 |
| 34 | 56 ± 11 | 0.02 ± 0.01 |

TB-Patient Assays. Based on these findings, an approach was followed to determine whether a lower LOD can improve the utility of LAM as an antigenic marker and therefore potentially advance TB diagnostics. Toward this end, 24 TB-positive (identifiers #1 to #24) and 10 healthy control (identifiers #25 to #34) serum specimens were analyzed.

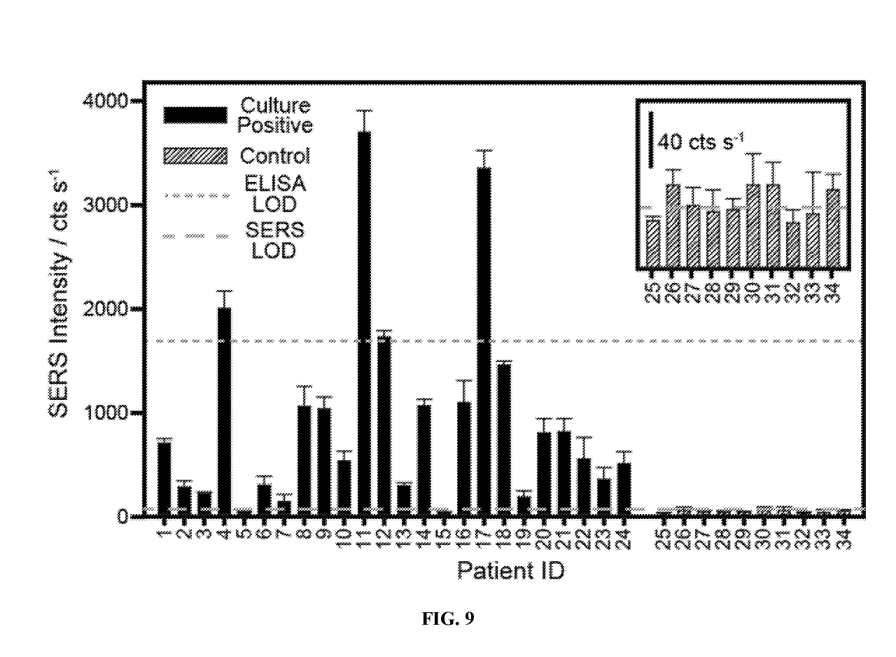

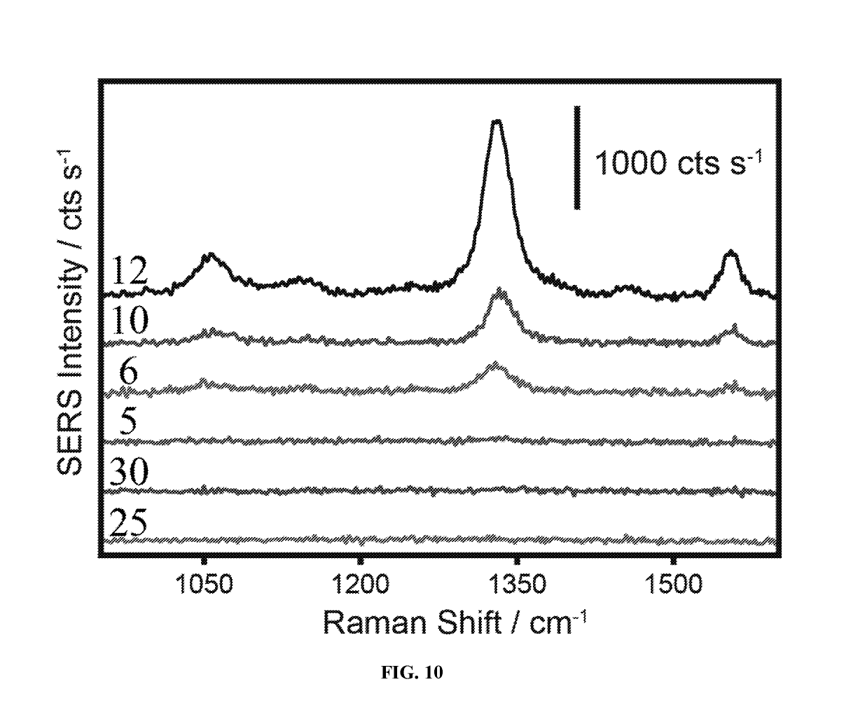

The results for the assays of the 34 different human serum specimens, after pretreatment, are presented in FIGS. 9 and 10. FIG. 9 shows the SERS analysis of patient serum (pretreated) for the quantification of LAM represented in a bar chart. The dashed-line delimiter represents the SERS LOD. The dotted-line delimiter represents the reported LOD for the ELISA test for LAM. The region between the two lines indicates the specimens with LAM levels detectable by SERS but potentially missed by conventional ELISA. The average SERS signal is calculated from the peak height of the vs(NO₂) from baseline corrected spectra, and all error bars represent the standard deviation of the response at ten different locations on duplicate samples. The LAM concentration scale for the vertical axis on the right hand side of the figure is constructed from the calibration plot in FIG. 7B.

FIG. 9 summarizes these measurements as histograms representing the average signal strength of the vs(NO₂) mode for each sample and LAM levels determined from the calibration plot in FIG. 7B. FIG. 9 also includes a delimiter for the LOD of the SERS assay (dashed line).

For illustrative purposes, a small set of specimen spectra is presented in FIG. 10. The data include spectra for two of the healthy control samples (#25 and #30) and four of the TB-positive samples (#5, #6, #10, and #12).

As evident from these data, LAM was found in 21 of the 24 TB-positive samples with analysis by SERS. It was not detectable in 3 of the TB-positive samples (i.e., #5, #7, and #15) or in any of the 10 healthy control specimens (i.e., LAM <10 pg/mL). Notably, the levels of LAM found in 17 of the TB-positive specimens were below, and in several cases well below, the reported LOD (˜1 ng/mL) of conventional ELISA for LAM.

Further inspection of these data draws out three other aspects of the results. First, a few of the TB-positive samples have comparatively high levels of LAM (#11 at 2.21(±0.12), #17 at 1.99(±0.10), #4 at 1.18(±0.10), and #12 at 1.02(±0.04 ng/mL)), all of which were in the range of what would be detectable by conventional ELISA. Most of the samples, however, had much lower amounts of measureable LAM (#21 at 0.47(±0.08), #10 at 0.30(±0.06), and #6 at 0.16(±0.05) ng/mL). One sample had a LAM level with a signal strength just above that needed to be statistically measurable (#19 at 0.09(±0.04) ng/mL) by the inventive methods. The ability to quantify small differences in LAM levels in TB-patient sera suggests that the inventive methods could be used to track the progression of the disease, monitor treatment responses, and potentially determine the optimal duration of therapy. All of these applications could also be integrated into assessments of new drug treatment regimens and/or vaccines.

These data also show that the responses for 3 of the TB-positive patient samples (#5, #7, and #15) were not distinguishable from those of the calibration blank or any of the controls. There was a hint of the presence for LAM in a few of the individual reading locations on sample #7 (not shown), but not at a level sufficiently persistent to be statistically valid when averaged over ten different locations on each of the duplicate runs. This could have resulted in a decreased bacterial burden to an undetectable level (note that the presence of anti-LAM antibodies found in the immunoblot reactivity tests of these specimens is only indicative of an immune response (past or present) by the patient to the infection but not necessarily the status of the infection).

While details regarding these TB-positive patient specimens with respect to the treatment regime are not available, the inability to detect LAM in these specimens may be attributed to one or a combination of at least four possibilities: (1) LAM was present in these 3 specimens at levels below the LOD of the assay approach; (2) these patients may have had a positive response to one of the drug treatments used in the TBTC-22 clinical trial; (3) these specimens may have degraded during storage and/or shipment prior to receipt or to freeze/thaw cycling when realiquoted for distribution; and (4) not all patients with cavitary TB necessarily have LAM circulating in their serum.

Finally, these data show that the responses for all 10 healthy control samples were commensurate with that of the serum blank used in the construction of the calibration plot. The spectra for sample #25 and #30 in FIG. 10 are representative of those for the remaining healthy control samples. The presence of nonspecific ERL adsorption in these samples even after increasing the signal acquisition time from 1 to 60 seconds was undetected.

Taken together, these results support the value of a SERS-based approach for the detection of cavitary TB, and for evaluating other types of patient specimens, including non-cavitary lung disease, TB patients co-infected with HIV, children and those with extrapulmonary infections. An obstacle in the detection of LAM in other body fluids (e.g., urine and cerebral spinal fluid) may be a consequence of very low concentrations of unbound antigen due to immunocomplexation. The novel pretreatment methods developed herein are useful in sample preparation for SERS as well as conventional ELISA and other diagnostic platforms.

### Example 5

### Use of the Pretreatment Methods for the Detection of LAM with ELISA

The success of pretreatment in the analysis of LAM in human serum with SERS was expanded to ELISA technology. Conventional ELISA has lacked the ability to detect LAM at low concentrations necessary in the detection of tuberculosis in patients. However, the analysis of LAM by ELISA after the samples have been exposed to the novel pretreatment methods described herein, have exhibited significantly lower detection limits than previously thought possible.

Aliquots of 700 μL of pooled human serum containing LAM at various concentrations were treated with perchloric acid, as described for Example 4. Next, the pretreated solutions were analyzed in an ELISA platform. Commercially available ELISA plates were modified with capture antibody specific to LAM and non-specific adsorption was minimized with a blocking agent, i.e., bovine serum albumin (BSA). The pretreated LAM solutions were run in triplicate on ELISA plates. The captured LAM was exposed to a secondary biotinylated LAM antibody, which was consequently tagged with streptavidin-horseradish peroxidase (HRP). The enzyme, HRP, then utilized an added substrate (tetramethylbenzidine) to produce a colored solution. The enzyme activity was quenched with sulfuric acid after a specified amount of time. Measuring the absorbance at a wavelength of 450 nm quantitated the amount of LAM captured.

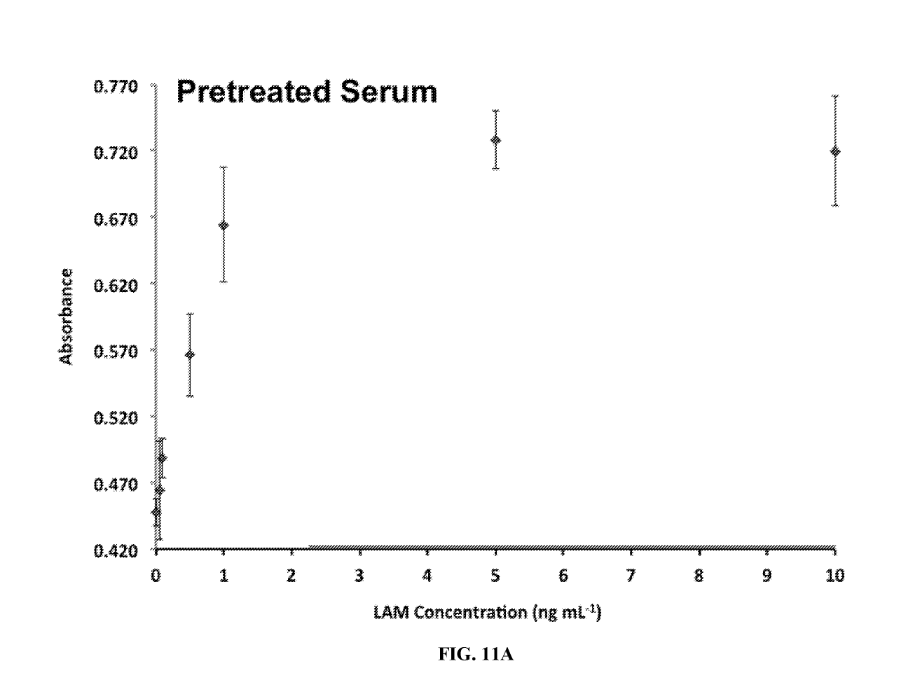

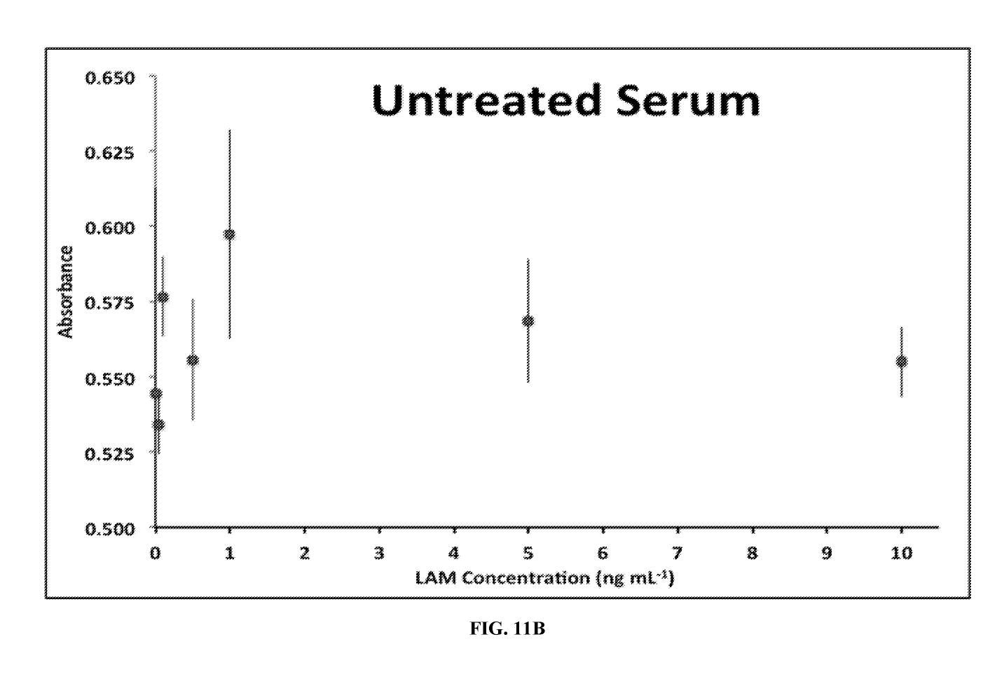

FIGS. 11A and 11B show dose-response curves for an ELISA-based immunoassay for LAM in human serum with perchloric acid pretreatment (FIG. 11A) and without pretreatment (FIG. 11B). The absorbance was measured at 450 nm. The average of 3 runs was plotted with error bars representing the standard deviation in the 3 measurements. The calculated LOD (i.e., blank signal plus three times the standard deviation) for LAM in human serum with pretreatment was 72 pg/mL.

Results from an ELISA detecting LAM in untreated serum is shown in FIG. 11B for comparison. The untreated (conventional) serum matrix does not allow for low-level detection of LAM, as LAM levels ranging from 25 pg/mL to 10 ng/mL yield the same response, indicating that LAM is masked in untreated serum. This data suggests that ELISA detection of LAM from human serum is only possible with the novel pretreatment methods disclosed herein.

### Example 6

### Comparative Example

A comparison of the novel pretreatment methods using complexing reagents with no or conventional pretreatment methods was performed, analyzing their ability to release LAM from complexing proteins and other components in human serum as a soluble product, following the procedures described in Example 4. The perchloric, trifluoroacetic, and sulfosalicylic acid pretreatment followed the method of Example 4 for perchloric acid, with the exception that the acid amounts were different for trifluoroacetic and sulfosalicylic acid, those being 7 μL (13 M) and 4 μL (2 M), respectively. After vortexing for 10 seconds and centrifuging at 12,045 g for 5 min, 75 μL of the resulting supernatant was transferred to a second centrifuge tube and neutralized to pH 7.5 with an aqueous solution of K₂CO₃ (2.0 M).

The conventional methanol pretreatment method mixed 200 μL of serum containing LAM with 200 μL of methanol. The solution was centrifuged at 12,045 g for 5 min. The supernatant (200 μL) was removed and heated at 70° C. for 20 min followed by another centrifugation step at 12,045 g for 5 min. This supernatant was consequently used in the SERS assay to detect LAM. The conventional heat pretreatment method was carried out by heating LAM spiked in human serum at 95° C. for 5 min followed by centrifugation at 12,045 g for 5 min. The supernatant was then used in the SERS assay to detect LAM.

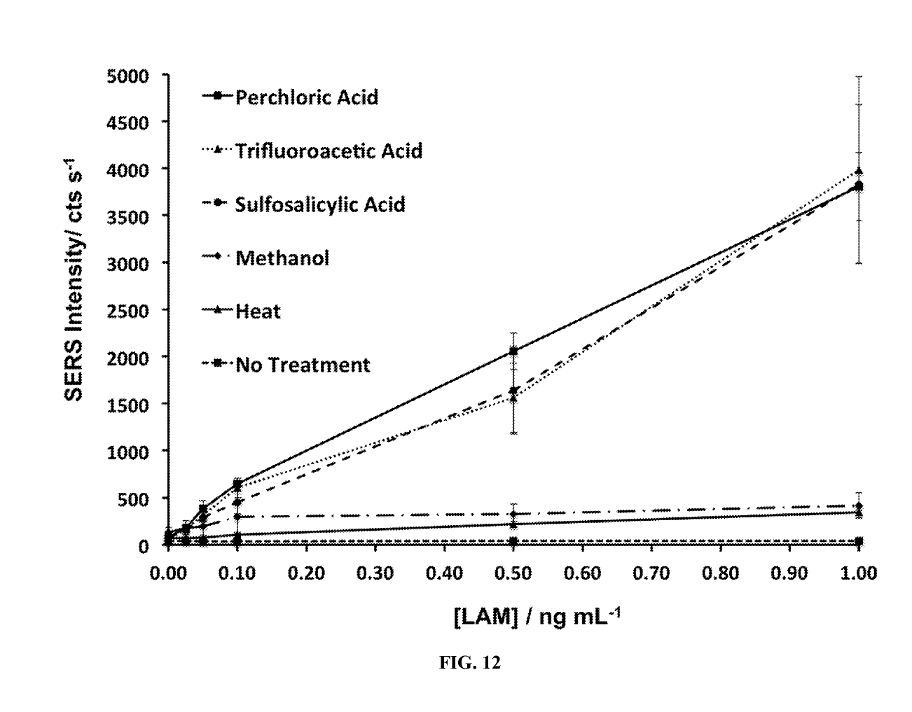

Aliquots of pooled human serum containing LAM at various concentrations were treated with various reagents in order to release LAM from complexation with constituents in human serum. Next, the pretreated solutions were analyzed in a SERS-based immunoassay as described in Example 4, and compared to LAM in human serum without pretreatment. Based on the raw Raman spectra, dose-response curves were constructed that plot SERS response as a function of LAM concentration. This is shown in FIG. 12 for the various pretreatments of LAM in human serum and for untreated solutions.

FIG. 12 shows dose-response curves for the results of a SERS-based immunoassay detecting LAM in human serum without pretreatment (squares with dashed line) and with pretreatment using heat (triangles with solid line), methanol (diamonds with dashed-dotted line), sulfosalicylic acid (circles with dashed line), trifluoroacetic acid (triangles with dotted line) or perchloric acid (squares with solid line). The values plotted for each curve represent the average of 10 spots on a single sample for each LAM concentration, and the associated error bars represent the standard deviation in the 10 measurements.

Notably, LAM could not be effectively detected without pretreatment and conventional pretreatments such as heat or organic solvents only resulted in minimal improvements in the detection of LAM in human serum. Acid pretreatment, such as perchloric acid addition, had a profound impact on the release of LAM from complexation with proteins and other components, and resulted in the detection of LAM at low levels in serum employing a SERS-based immunoassay.

Accordingly, methods for detecting LAM in a biological sample can be performed using a variety of analytical detection platforms. The methods may include contacting the biological sample with an acid selected from the group consisting of perchloric acid, trifluoroacetic acid, and sulfosalicylic acid. The methods may also include removing protein precipitate and/or complexes from the biological sample after contacting the biological sample with an acid. Such protein precipitate may be removed by centrifugation. The methods may also include determining the LAM concentration in the biological sample after removing the protein precipitate.

These methods for detecting LAM in a biological sample may be useful for detecting a variety of diseases, such as for diagnosing tuberculosis in a mammal. Such a method would include contacting a serum sample of the mammal with an acid selected from the group consisting of perchloric acid, trifluoroacetic acid, and sulfosalicylic acid. These diagnostic methods could also include removing protein precipitate and/or complexes from the biological sample after contacting the biological sample with an acid, and determining the LAM concentration in the serum sample.

The types of analytical detection platforms which may be used to detect analytes with the methods disclosed herein include essentially any assay or analytical process which provides a signal or response to a change or the presence (or absence) of an analyte. These platforms include, for example, ELISA, surface-enhanced Raman scattering (SERS)-based immunoassay, any assay using detection via fluorescence, diffuse reflectance, mass spectrometric, liquid or gas chromatographic spectroscopies, any magnetic, colorometric or electrochemical based detection platform, lateral and vertical flow assays, and kinetics-based assays such as surface plasmon resonance. The biological samples used in these methods include mammalian serum. The concentrations of the analytes detected in the biological sample using the novel pretreatment methods range from about 0.01 to about 10,000 ng/mL. The methods for detecting LAM in a biological sample can be performed with a kit which includes instructions for its use.

### REFERENCES

Each of the following citations is fully incorporated herein by reference in its entirety.
1. Phillips, L., Infectious disease: TB's revenge. Nature, 2013. 493(7430): p. 14.
2. World Health Organization, Global Tuberculosis Report 2013. 2013.
3. Zumla, A., et al., Tuberculosis. New Engl. J. of Medicine, 2013. 368(8): p. 745-755.
4. Steingart, K. R., et al., Sputum processing methods to improve the sensitivity of smear microscopy for tuberculosis: A systematic review. The Lancet Infectious Diseases, 2006. 6(10): p. 664-674.
5. Steingart, K. R., et al., Commercial serological tests for the diagnosis of active pulmonary and extrapulmonary tuberculosis: An updated systematic review and meta-analysis. PLOS Medicine, 2011. 8(8): p. e1001062.
6. Patel, V. B., et al., Comparison of a clinical prediction rule and a LAM antigen-detection assay for the rapid diagnosis of TBM in a high HIV prevalence setting. PLOS ONE, 2010. 5(12): p. e15664.
7. Perkins, M. D. and J. Cunningham, Facing the crisis: Improving the diagnosis of tuberculosis in the HIV era. J. of Infectious Diseases, 2007. 196(Supplement 1): p. S15-S27.
8. Theron, G., et al., Do adjunct tuberculosis tests, when combined with Xpert MTB/RIF, improve accuracy and the cost of diagnosis in a resource-poor setting? European Respiratory Journal, 2012. 40(1): p. 161-168.
9. Liong, M., et al., Magnetic barcode assay for genetic detection of pathogens. Nature Communications, 2013. 4: p. 1752.
10. Abebe, F., et al., Progress in serodiagnosis of Mycobacterium tuberculosis infection. Scandinavian Journal of Immunology, 2007. 66(2-3): p. 176-191.
11. Reither, K., et al., Low sensitivity of a urine LAM-ELISA in the diagnosis of pulmonary tuberculosis. BMC Infectious Diseases, 2009. 9(1): p. 141.
12. World Health Organization, Malaria Rapid Diagnostic Test Performance. 2010.
13. Chatterjee, D. and K. H. Khoo, Mycobacterial lipoarabinomannan: An extraordinary lipoheteroglycan with profound physiological effects. Glycobiology, 1998. 8(2): p. 113-120.
14. Lawn, S. D., Point-of-care detection of lipoarabinomannan (LAM) in urine for diagnosis of HlVassociated tuberculosis: A state of the art review. BMC Infectious Diseases, 2012. 12(1): p. 103.
15. Sarkar, S., et al., A bispecific antibody based assay shows potential for detecting tuberculosis in resource constrained laboratory settings. PLOS ONE, 2012. 7(2): p. e32340.
16. Hamasur, B., Rapid diagnosis of tuberculosis by detection of mycobacterial lipoarabinomannan in urine. Journal of Microbiological Methods, 2001. 45(1): p. 41-52.
17. Chan, J., Lipoarabinomannan, a possible virulence factor involved in persistence of M. tuberculosis within macrophages. Infection and Immunity, 1991. 59(5): p. 1755-1761.
18. Arias-Bouda, L. M. P., et al., Development of antigen detection assay for diagnosis of tuberculosis using sputum samples. J. of Clinical Microbiology, 2000. 38(6): p. 2278-2283.
19. Lyashchenko, K. P., et al., PrimaTB STAT-PAK Assay, a Novel, Rapid Lateral-Flow Test for Tuberculosis in Nonhuman Primates. Clinical and Vaccine Immunology, 2007. 14(9): p. 1158-1164.
20. Daley, P., Blinded evaluation of commercial urinary lipoarabinomannan for active tuberculosis: A pilot study. International Journal of Tuberculosis and Lung Disease, 2009. 13(8): p. 989-995.
21. Dheda, K., et al., Clinical utility of a commercial LAM-ELISA assay for TB diagnosis in HIV-infected patients using urine and sputum samples. PLOS ONE, 2010. 5(3): p. e9848.
22. Minion, J., et al., Diagnosing tuberculosis with urine lipoarabinomannan: Systematic review and meta-analysis. European Respiratory Journal, 2011. 38(6): p. 1398-1405.
23. Granger, J., et al., Toward development of a surface-enhanced Raman scattering (SERS)-based cancer diagnostic immunoassay panel. Analyst, 2013. 138(2): p. 410-416.
24. Driskell, J. D., Low-level detection of viral pathogens by a surface-enhanced Raman scattering based immunoassay. Analytical Chemistry, 2005. 77(19): p. 6147-6154.
25. Grubisha, D. S., Femtomolar detection of prostate-specific antigen: An immunoassay based on surface-enhanced Raman scattering and immunogold labels. Analytical Chemistry, 2003. 75(21): p. 5936-5943.
26. Stuart, D. A., et al., Biological applications of localised surface plasmonic phenomenae. IEE Proceedings—Nanobiotechnology, 2005. 152(1): p. 13-32.
27. Xu, S., et al., Immunoassay using probe-labelling immunogold nanoparticles with silver staining enhancement via SERS. Analyst, 2004. 129(1): p. 63-68.
28. Chon, H., et al., Highly Sensitive Immunoassay of Lung Cancer Marker Carcinoembryonic Antigen Using Surface-Enhanced Raman Scattering of Hollow Gold Nanospheres. Analytical Chemistry, 2009. 81(8): p. 3029-3034.
29. Bantz, K., et al., Recent progress in SERS biosensing. Physical Chemistry Chemical Physics, 2011. 13(24): p. 11551-11567.
30. Graham, D. and R. Goodacre, Chemical and bioanalytical applications of surface enhanced Raman scattering spectroscopy. Chem. Society Reviews, 2008. 37(5): p. 883-884.
31. Porter, M. D., et al., SERS as a bioassay platform: Fundamentals, design, and applications. Chemical Society Reviews, 2008. 37(5): p. 1001-1011.
32. Sharma, B., et al., SERS: Materials, applications, and the future. Materials Today, 2012. 15(1-2): p. 16-25.
33. Moskovits, M., Surface-enhanced spectroscopy. Reviews of Modern Physics, 1985. 57(3): p. 783-826.
34. McCreery, R. L., Raman spectroscopy for chemical analysis. Vol. 225. 2005: Wiley-Interscience.
35. Driskell, J., R Lipert, and M. Porter, Labeled Gold Nanoparticles Immobilized at Smooth Metallic Substrates: Systematic Investigation of Surface Plasmon Resonance and Surface-Enhanced Raman Scattering. The Journal of Physical Chemistry. B, 2006. 110(35): p. 17444-17451.
36. Haiss, W., et al., Determination of size and concentration of gold nanoparticles from UV-Vis spectra. Analytical Chemistry, 2007. 79(11): p. 4215-4221.
37. Bradley, M. Manuscript in Preparation.
38. Hegner, M., Ultralarge atomically flat template-stripped Au surfaces for scanning probe microscopy. Surface Science, 1993. 291(1-2): p. 39-46.
39. Park, H.-Y., et al., Surface-enhanced Raman scattering: physics and applications. Vol. 103. 2006: Springer. 427-446.
40. Gaylord, H., Most M. leprae carbohydrate-reactive monoclonal antibodies are directed to lipoarabinomannan. Infection and Immunity, 1987. 55(11): p. 2860-2863.
41. Tessema, T., et al., Circulating antibodies to lipoarabinomannan in relation to sputum microscopy, clinical features and urinary anti-lipoarabinomannan detection in pulmonary tuberculosis. Scandinavian Journal of Infectious Diseases, 2002. 34(2): p. 97-103.
42. Sakamuri, R., et al., Association of lipoarabinomannan with high density lipoprotein in blood: Implications for diagnostics. Tuberculosis, 2013. 93(3): p. 301-307.
43. Scopes, R. K., Protein purification: principles and practice. 1994: Springer.
44. Crawford, A. Manuscript in Preparation.
45. Anderson, A. J., Factors affecting the amount and composition of the serum seromucoid fraction. Nature, 1965. 208(5009): p. 491-492.
46. Benator, D., Rifapentine and isoniazid once a week versus rifampicin and isoniazid twice a week for treatment of drug-susceptible pulmonary tuberculosis in HIV-negative patients: A randomized clinical trial. Lancet, 2002. 360(9332): p. 528-534.
47. Spencer, J. S., et al., Analysis of antibody responses to Mycobacterium leprae phenolic glycolipid I, lipoarabinomannan, and recombinant proteins to define disease subtype-specific antigenic profiles in leprosy. Clinical and Vaccine Immunology, 2011. 18(2): p. 260-267.
48. Patil, S., G. Ramu, and R. Prasad, Detection of disease related immune complexes in the serum of leprosy patients. Journal of Neuroimmunology, 2000. 105(1): p. 64-68.
49. Sada, E., et al., Detection of lipoarabinomannan as a diagnostic test for tuberculosis. Journal of Clinical Microbiology, 1992. 30(9): p. 2415-2418.
50. Schlesinger, L. S., Binding of the terminal mannosyl units of lipoarabinomannan from a virulent strain of Mycobacterium tuberculosis to human macrophages. The Journal of Immunology, 1994. 152(8): p. 4070-4079.
51. Stynen, D., A new sensitive sandwich enzyme-linked immunosorbent assay to detect galactofuran in patients with invasive Aspergillosis. Journal of Clinical Microbiology, 1995. 33(2): p. 497-500.
52. Layne, E., Spectrophotometric and turbidimetric methods for measuring proteins. Methods in Enzymology, 1957. 3(C): p. 447-454.
53. Stoscheck, C. and M. P. Deutscher, Quantitation of protein: Guide to Protein Purification. Methods in Enzymilogy, 1990. 182: p. 50-68.
54. Mdivani, N., et al., Monitoring Therapeutic Efficacy by Real-Time Detection of M. tuberculosis mRNA in Sputum. Clinical Chemistry, 2009. 55(9): p. 1694-1700.
55. Jiang, L., Rapid Detection and Monitoring Therapeutic Efficacy of Mycobacterium tuberculosis Complex Using a Novel Real-Time Assay. Journal of Microbiology and Biotechnology, 2012. 22(9): p. 1301-1306.
56. Yew, W. W., Therapeutic drug monitoring in antituberculosis chemotherapy: clinical perspectives. Clinica Chimica Acta, 2001. 313(1-2): p. 31-36.
57. Keeler, E., et al., Reducing the global burden of tuberculosis: the contribution of improved diagnostics. Nature, 2006. 444: p. 49-57.
58. Mukundan, H., et al., Rapid detection of Mycobacterium tuberculosis biomarkers in a sandwich immunoassay format using a waveguide-based optical biosensor. Tuberculosis, 2012. 92(5): p. 407-416.
59. Schmidt, R., et al., Single-molecule detection on a protein-array assay platform for the exposure of a tuberculosis antigen. Journal of Proteome Research, 2011. 10(3): p. 1316-1322.
60. Park, H.-Y., et al., Single-particle Raman measurements of gold nanoparticles used in surface enhanced Raman scattering (SERS)-based sandwich immunoassays. Proceedings of SPIE—the International Society for Optical Engineering, 2004. 5593: p. 464-477.
61. Rietschel, E. T.; Kirikae, T.; Schade, F. U.; Mamat, U.; Schmidt, G.; Loppnow, H.; Ulmer, A. J.; Zahringer, U.; Seydel, U.; Di Padova, F., Bacterial endotoxin: molecular relationships of structure to activity and function. The FASEB Journal 1994, 8 (2), 217.
62. Hurley, J. C., Endotoxemia: methods of detection and clinical correlates. Clinical Microbiology Reviews 1995, 8 (2), 268.
63. Beutler, B.; Rietschel, E. T., Innate immune sensing and its roots: the story of endotoxin. Nat Rev Immunol 2003, 3 (2), 169.
64. Van Pittius, N. G.; Gamieldien, J.; Hide, W.; Brown, G. D.; Siezen, R. J.; Beyers, A. D., The ESAT-6 gene cluster of Mycobacterium tuberculosis and other high G+ C Gram-positive bacteria. Genome Biol 2001, 2 (10), 44.1.
65. Barnes, P. F.; Chatterjee, D.; Abrams, J. S.; Lu, S.; Wang, E.; Yamamura, M.; Brennan, P. J.; Modlin, R. L., Cytokine production induced by M. tuberculosis lipoarabinomannan. Relationship to chemical structure. The J. of Immun. 1992, 149 (2), 541.
66. Elass, E.; Coddeville, B.; Guerardel, Y.; Kremer, L.; Maes, E.; Mazurier, J.; Legrand, D., Identification by surface plasmon resonance of the mycobacterial lipomannan and lipoarabinomannan domains involved in binding to CD14 and LPSbinding protein. FEBS Letters 2007, 581 (7), 1383.
67. Lodowska, J.; Wolny, D.; Weglarz, L., The sugar 3-deoxy-d-manno-oct-2-ulosonic acid (Kdo) as a characteristic component of bacterial endotoxin—a review of its biosynthesis, function, and placement in the lipopolysaccharide core. Canadian Journal of Microbiology 2013, 59 (10), 645.
68. Trent, M. S.; Stead, C. M.; Tran, A. X.; Hankins, J. V., Invited review: Diversity of endotoxin and its impact on pathogenesis. Journal of Endotoxin Research 2006, 12 (4), 205.
69. Ellwood, D. C., The Distribution of 2-Keto-3-deoxy-octonic Acid in Bacterial Walls. Journal of General Microbiology 1970, 60 (3), 373.
70. Fukuda, T.; Matsumura, T.; Ato, M.; Hamasaki, M.; Nishiuchi, Y.; Murakami, Y.; Maeda, Y.; Yoshimori, T.; Matsumoto, S.; Kobayashi, K.; Kinoshita, T.; Morita, Y. S., Critical roles for lipomannan and lipoarabinomannan in cell wall integrity of mycobacteria and pathogenesis of tuberculosis. mBio 2013, 4 (1), e00472.
71. Mishra, A. K.; Driessen, N. N.; Appelmelk, B. J.; Besra, G. S., Lipoarabinomannan and related glycoconjugates: structure, biogenesis and role in Mycobacterium tuberculosis physiology and host-pathogen interaction. FEMS Microbiology Reviews 2011, 35 (6), 1126.
72. Chatterjee, D., The mycobacterial cell wall: structure, biosynthesis and sites of drug action. Current Opinion in Chemical Biology 1997, 1 (4), 579.
73. Karima, R.; Matsumoto, S.; Higashi, H.; Matsushima, K., The molecular pathogenesis of endotoxic shock and organ failure. Molecular Medicine Today 1999, 5 (3), 123.
74. Guerardel, Y.; Maes, E.; Elass, E.; Leroy, Y.; Timmerman, P.; Besra, G. S.; Locht, C.; Strecker, G.; Kremer, L., Structural study of lipomannan and lipoarabinomannan from Mycobacterium chelonae. Presence of unusual components with alpha 1,3-mannopy ranose side chains. The Journal of Biological Chemistry 2002, 277 (34), 30635.
75. Krzyzowska, M.; Schollenberger, A.; Pawlowski, A.; Hamasur, B.; Winnicka, A.; Augustynowicz-Kopec, E.; Niemialtowski, M., Lipoarabinomannan as a regulator of the monocyte apoptotic response to Mycobacterium bovis BCG Danish strain 1331 infection. Polish journal of microbiology/Polskie Towarzystwo Mikrobiologow= The Polish Society of Microbiologists 2007, 56 (2), 89.
76. Tanaka, S.; Takahashi, S. Method of Eliminating Reactivity of Lipoarabinomannan and Application of the Same. 2009.
77. Petsch, D.; Anspach, F. B., Endotoxin removal from protein solutions. Journal of Biotechnology 2000, 76 (2-3), 97.
78. Sharma, S. K., Endotoxin detection and elimination in biotechnology. Biotechnology and Applied Biochemistry 1986, 8 (1), 5.
79. Atha, D. H.; Ingham, K. C., Mechanism of precipitation of proteins by polyethylene glycols. Analysis in terms of excluded volume. J. of Biol. Chemistry 1981, 256 (23), 12108.
80. Ingham, K. C., Precipitation of proteins with polyethylene glycol. Methods in Enzymology 1990, 182, 301.
81. Bhat, R.; Timasheff, S. N., Steric exclusion is the principal source of the preferential hydration of proteins in the presence of polyethylene glycols. Protein Sci. 1992, 1 (9), 1133.
82. Raja, A.; Narayanan, P.; Mathew, R.; Prabhakar, R., Characterization of mycobacterial antigens and antibodies in circulating immune complexes from pulmonary tuberculosis. The Journal of Laboratory and Clinical Medicine 1995, 125 (5), 581.
83. Carr, R.; Chakraborty, A.; Brunda, M.; Davidson, P.; Damle, P.; Hardtke, M.; Gilbride, K.; Minden, P., Immune complexes and antibodies to BCG in sera from patients with mycobacterial infections. Clinical and Experimental Immunology 1980, 39 (3), 562.
84. Samuel, A.; Ashtekar, M.; Ganatra, R., Significance of circulating immune complexes in pulmonary tuberculosis. Clinical and Experimental Immunology 1984, 58 (2), 317.
85. Bhattacharya, A.; Ranadive, S.; Kale, M.; Bhattacharya, S., Antibody based enzyme linked immunosorbent assay for determination of immune-complexes in clinical tuberculosis. American Journal of Respiratory and Critical Care Medicine 1986, 134 (2), 205.
86. Udaykumar; Sarin, R.; Saxena, R. K., Analysis of circulating immune complexes (CIC) in tuberculosis: Levels of specific antibody and antigens in CIC and relationship with serum antibody. FEMS Microbiology Letters 1991, 76 (3), 135.
87. Radhakrishnan, V.; Mathai, A.; Sundaram, P., Diagnostic significance of circulating immune complexes in patients with pulmonary tuberculosis. Journal of Medical Microbiology 1992, 36 (2), 128.
88. Raja, A.; Devi, K. U.; Ramalingam, B.; Brennan, P. J., Immunoglobulin G, A, and M responses in serum and circulating immune complexes elicited by the 16-kilodalton antigen of M. tuberculosis. Clinical and Diagnostic Laboratory Immunology 2002, 9 (2), 308.
89. Mehta, P. K.; Khuller, G. K., Comparative evaluation of the diagnostic significance of circulating immune complexes and antibodies to phosphatidylinositomannosides in pulmonary tuberculosis by enzyme-linked immunosorbent assay. Medical Microbiology and Immunology 1989, 178 (4), 229.
90. Mennink-Kersten, M. A. S. H.; Donnelly, J. P.; Verweij, P. E., Detection of circulating galactomannan for the diagnosis and management of invasive aspergillosis. The Lancet Infectious Diseases 2004, 4 (6), 349.
91. Doskeland, S.; Berdal, B., Bacterial antigen detection in body fluids: methods for rapid antigen concentration and reduction of nonspecific reactions. Journal of Clinical Microbiology 1980, 11 (4), 380.
92. Maruyama, C. Immunotherapeutic Agent for Tumors Comprising Lipopolysaccharide as an Active Component. 1983.
93. Gillespie, S. H.; Smith, M. D.; Dickens, A.; Raynes, J. G.; McAdam, K. P., Detection of C-polysaccharide in serum of patients with Streptococcus pneumonia bacteraemia. Journal of Clinical Pathology 1995, 48 (9), 803.
94. Troseid, M.; Nowak, P.; Nystrom, J.; Lindkvist, A.; Abdurahman, S.; Sonnerborg, A., Elevated plasma levels of lipopolysaccharide and high mobility group box-1 protein are associated with high viral load in HIV-1 infection: reduction by 2-year antiretroviral therapy. AIDS 2010, 24 (11), 1733.
95. Troseid, M.; Lind, A.; Nowak, P.; Barqasho, B.; Heger, B.; Lygren, I.; Pedersen, K. K.; Kanda, T.; Funaoka, H.; Damas, J. K.; Kvale, D., Circulating levels of HMGB1 are correlated strongly with MD2 in HIV-infection: Possible implication for TLR4-signalling and chronic immune activation. Innate Immunity 2013, 19 (3), 290.
96. Feruglio, S. L.; Troseid, M.; Damas, J. K.; Kvale, D.; Dyrhol-Riise, A. M., Soluble Markers of the Toll-Like Receptor 4 Pathway Differentiate between Active and Latent Tuberculosis and Are Associated with Treatment Responses. PLoS ONE 2013, 8 (7), e69896.
97. Obayashi, T., Addition of perchloric acid to blood samples for colorimetric limulus test using chromogenic substrate: comparison with conventional procedures and clinical applications. J Lab Clin Med 1984, 104 (3), 321.
98. Obayashi, T.; Tamura, H.; Tanaka, S.; Ohki, M.; Takahashi, S.; Kawai, T., Endotoxin-inactivating activity in normal and pathological human blood samples. Infection and Immunity 1986, 53 (2), 294.
99. De Jonge, N.; Fillie, Y. E.; Deelder, A. M., A simple and rapid treatment (trichloroacetic acid precipitation) of serum samples to prevent non-specific reactions in the immunoassay of a proteoglycan. Journal of Immunological Methods 1987, 99 (2), 195.
100. Inada, K.; Endo, S.; Takahashi, K.; Suzuki, M.; Narita, T.; Yoshida, T.; Suda, H.; Komuro, T.; Yoshida, M., Establishment of a new perchloric acid treatment method to allow determination of the total endotoxin content in human plasma by the limulus test and clinical application. Microbiology and Immunology 1991, 35 (4), 303.
101. Suda, H.; Moroi, C.; Inada, K.; Chida, S.; Yoshida, M., Application of a new perchloric acid treatment method to measure endotoxin in both amniotic fluid and cord blood by an endotoxin-specific chromogenic Limulus test in intra-amniotic infection. Acta Paediatrica Japonica 1996, 38 (5), 444.
102 Petsch, D.; Deckwer, W. D.; Anspach, F. B., Proteinase K digestion of proteins improves detection of bacterial endotoxins by the Limulus amebocyte lysate assay: application for endotoxin removal from cationic proteins. Analytical Biochemistry 1998, 259 (1), 42.

Various features and advantages of the invention are set forth in the following claims.

1. A method for detecting lipoarabinomannan in a biological sample, the method comprising contacting the biological sample with an acid selected from the group consisting of perchloric acid, trifluoroacetic acid, and sulfosalicylic acid.

2. The method of claim 1, further comprising removing protein precipitate from the biological sample after contacting the biological sample with the acid.

3. The method of claim 2, wherein the protein precipitate is removed by centrifugation.

4. The method of claim 2, further comprising determining the lipoarabinomannan concentration in the biological sample after removing the protein precipitate.

5. The method of claim 4, wherein the lipoarabinomannan concentration is determined using at least one of ELISA; a surface-enhanced Raman scattering (SERS)-based immunoassay; an assay using detection via fluorescence, diffuse reflectance, mass spectrometric, liquid or gas chromatographic spectroscopies; a magnetic, colorometric or electrochemical response; a lateral or vertical flow assay; and surface plasmon resonance.

6. The method of claim 1, wherein the biological sample comprises a serum of a mammal.

7. The method of claim 1, wherein the method is capable of detecting lipoarabinomannan in the biological sample at concentrations of from about 0.01 to about 10,000 ng/mL.

8. A kit for detecting lipoarabinomannan in a biological sample using the method of claim 1, the kit comprising an acid selected from the group consisting of perchloric acid, trifluoroacetic acid, and sulfosalicylic acid, and further comprising instructions for contacting the acid with a biological sample.

9. A method for diagnosing tuberculosis in a mammal, the method comprising contacting a serum sample of the mammal with an acid selected from the group consisting of perchloric acid, trifluoroacetic acid, and sulfosalicylic acid.

10. The method of claim 9, further comprising removing protein precipitate from the serum sample after contacting the serum sample with the acid, and determining the lipoarabinomannan concentration in the serum sample.

11. The method of claim 10, wherein the lipoarabinomannan concentration is determined using at least one of surface-enhanced Raman scattering (SERS)-based immunoassay and ELISA.
